(19) United States
(12) Patent Application Publication
(10) Pub. No.: US 2023/0047787 A1
(43) Pub. Date: Feb. 16, 2023

(54) CONTROLLING PROGRESS OF AUDIO-VIDEO CONTENT BASED ON SENSOR DATA OF MULTIPLE USERS, COMPOSITE NEURO-PHYSIOLOGICAL STATE AND/OR CONTENT ENGAGEMENT POWER

(71) Applicant: Warner Bros. Entertainment Inc., Burbank, CA (US)

(72) Inventors: Arvel Chappell, III, La Mirada, CA (US); Michael Zink, Altadena, CA (US); Ha Nguyen, Fullerton, CA (US); Clayton Mosher, Los Angeles, CA (US); Andrew Veeder, Los Angeles, CA (US); Michael Martinez, Redondo Beach, CA (US); Shanshan Sun, Chino Hills, CA (US); Gary Lake-Schaal, Alameda, CA (US)

(21) Appl. No.: 17/963,741

(22) Filed: Oct. 11, 2022

Related U.S. Application Data
(63) Continuation-in-part of application No. 16/923,033, filed on Jul. 7, 2020.
Application No. 16/923,033 is a continuation of application No. PCT/US2019/012567, filed on Jan. 7, 2019.
(60) Provisional application No. 62/715,766, filed on Aug. 7, 2018.
(60) Provisional application No. 62/661,556, filed on Apr. 23, 2018.
(60) Provisional application No. 62/614,811, filed on Jan. 8, 2018.

(51) Int. Cl. G06F 3/01

(52) U.S. Cl. CPC G06F 3/015; G06F 2203/011

(57) ABSTRACT

Provided is a system for controlling progress of audio-video content based on sensor data of multiple users, composite neuro-physiological state (CNS) and/or content engagement power (CEP). Sensor data is received from sensors positioned on an electronic device of a first user to sense neuro-physiological responses of the first user and second users that are in field-of-view (FOV) of the sensors. Based on the sensor data and at least one of a CNS value for social interaction application and a CEP value for immersive content, recommendations of action items for first user are predicted. Content of a feedback loop, created based on sensor data, CNS value, CEP value, and predicted recommendations, is rendered on output unit of electronic device during play of the at least one of social interaction application and immersive content experience. Progress of social interaction and immersive content experience is controlled by first user based on predicted recommendations.

### CROSS-REFERENCE TO RELATED APPLICATION

The present application is a continuation-in-part of U.S. patent application Ser. No. 16/923,033, filed on Jul. 7, 2020, which is a continuation of international (PCT) application No. US2019012567 filed Jan. 7, 2019, which claims priority to United States Provisional Patent Application Serial Nos. 62/614,811, filed on Jan. 8, 2018, 62/661,556 filed on Apr. 23, 2018, and 62/715,766 filed on Aug. 7, 2018, which applications are incorporated herein by reference in their entireties.

### FIELD

The present disclosure relates to applications, methods and apparatus for signal processing of biometric sensor data from detection of neuro-physiological state in communication enhancement and game applications.

### BACKGROUND

Many humans are adept at empathizing feelings of others during communication; others less so. As electronic mediums have become increasingly used commonly for much interpersonal or human-machine communication, emotional signaling through visible and audible cues has become more difficult or impossible between people using electronic communication media. In text media, users resort to emojis or other manual signals. Often, emotional communication fails, and users misunderstand one another's intent. In addition, some people are adept at disguising their feelings, and sometimes use their skills to deceive or mislead others. Equipment such as lie detectors is used to address these problems in limited contexts but is too cumbersome and intrusive for widespread use. Further, advanced electronic components, such as sensors, equipped in new age electronic devices are further enhancing the utility of such electronic devices as never before. However, such electronic devices are used in a one-directional mode to sense the emotional signals of the corresponding users who are using or wearing such electronic devices.

In a related problem, many computer games are unresponsive to the user's emotional signals, which may cause players to lose interest in game play over time.

It would be desirable, therefore, to develop new methods and other new technologies for enhanced interpersonal or human-machine communication and games, that overcome these and other limitations of the prior art and help producers deliver more compelling entertainment experiences for the audiences of tomorrow.

### SUMMARY

This summary and the following detailed description should be interpreted as complementary parts of an integrated disclosure, which parts may include redundant subject matter and/or supplemental subject matter. An omission in either section does not indicate priority or relative importance of any element described in the integrated application. Differences between the sections may include supplemental disclosures of alternative embodiments, additional details, or alternative descriptions of identical embodiments using different terminology, as should be apparent from the respective disclosures. A previous application, Ser. No. 62/661,556 filed Apr. 23, 2018, lays a foundation for digitally representing user engagement with audio-video content, including but not limited to digital representation of Content Engagement Power (CEP) based on the sensor data, similar to Composite Neuro-physiological State (CNS) described in the present application. As described more fully in the earlier application, a computer process develops CEP for content based on sensor data from at least one sensor positioned to sense an involuntary response of one or more users while engaged with the audio-video output. For example, the sensor data may include one or more of electroencephalographic (EEG) data, galvanic skin response (GSR) data, facial electromyography (fEMG) data, electrocardiogram (EKG) data, video facial action unit (FAU) data, brain machine interface (BMI) data, video pulse detection (VPD) data, pupil dilation data, functional magnetic resonance imaging (fMRI) data, body chemical sensing data and functional near-infrared data (fNIR) received from corresponding sensors. The same or similar sensors may be used for calculation of CNS. “User” means an audience member, a person experiencing a video game or other application facilitating social interaction as a consumer for entertainment purposes. The present application builds on that foundation, making use of CNS in various applications summarized below.

CNS is an objective, algorithmic and digital electronic measure of a user's biometric state that correlates to a neuro-physiological state of the user during social interaction, for example while playing a video game or participating in an application facilitating social interaction. As used herein, “social interaction” includes any game in which two or more people interact, and other forms of social interaction such as interpersonal communication or simulated social interaction as when a user plays against a non-player character operated by a computer or against (e.g., in comparison with) prior performances by herself. In a given social interaction, the user may be concerned with learning how an inner neuro-physiological state corresponds to an outward effect detectable by sensors. The state of interest may be the user's own neuro-physiological state, or that of another user. As used herein, “neuro-physiological” means indicating or originating from a person's physiological state, neurological state, or both states. “Biometric” means a measure of a biological state, which encompasses “neuro-physiological” and may encompass other information, for example, identity information. Some data, for example, images of people's faces or other body portions, may indicate both identity and neuro-physiological state. As used herein, “biometric” always includes “neuro-physiological”.

CNS expresses at least two orthogonal measures, for example, arousal and valence. As used herein, “arousal” means a state or condition of being physiologically alert, awake and attentive, in accordance with its meaning in psychology. High arousal indicates interest and attention, low arousal indicates boredom and lack of interest. “Valence” is also used here in its psychological sense of attractiveness or goodness. Positive valence indicates attraction, and negative valence indicates aversion.

In an aspect, a method for controlling a social interaction application based on a representation (e.g., a quantitative measure or a qualitative symbol) of a neuro-physiological state of a user may include monitoring, by at least one processor, digital data from a social interaction, e.g., a game or unstructured chat. The method may include receiving sensor data from at least one sensor positioned to sense a neuro-physiological response of at least one user during the social interaction. The method may include determining a Composite Neuro-physiological State (CNS) value, based on the sensor data and recording the CNS value in a computer memory and/or communicating a representation of the CNS value to the user, or to another participant in the social interaction. In an aspect, determining the CNS value may further include determining arousal values based on the sensor data and comparing a stimulation average arousal based on the sensor data with an expectation average arousal. The sensor data may include one or more of electroencephalographic (EEG) data, galvanic skin response (GSR) data, facial electromyography (fEMG) data, electrocardiogram (EKG) data, video facial action unit (FAU) data, brain machine interface (BMI) data, video pulse detection (VPD) data, pupil dilation data, functional magnetic resonance imaging (fMRI) data, and functional near-infrared data (fNIR).

In an aspect, calculating a Composite Neuro-physiological State (CNS) may be based on the cognitive appraisal model. In addition, calculating the CNS value may include determining valence values based on the sensor data and including the valence values in determining the measure of a neuro-physiological state. Determining valence values may be based on sensor data including one or more of electroencephalographic (EEG) data, facial electromyography (fEMG) data, video facial action unit (FAU) data, brain machine interface (BMI) data, functional magnetic resonance imaging (fMRI) data, functional near-infrared data (fNIR), and positron emission tomography (PET).

In a related aspect, the method may include determining the expectation average arousal based on further sensor data measuring a like involuntary response of the recipient while engaged with known audio-video stimuli. Accordingly, the method may include playing the known audio-video stimuli comprising a known non-arousing stimulus and a known arousing stimulus. More detailed aspects of determining the CNS value, calculating one of multiple event powers for each of the one or more users, assigning weights to each of the event powers based on one or more source identities for the sensor data, determining the expectation average arousal and determining valence values based on the sensor data may be as described for other application, herein above or in the more detailed description below.

In an aspect, a method for controlling progress of audio-video content based on sensor data of multiple users, composite neuro-physiological state and/or content engagement power may include monitoring receiving sensor data from one or more sensors positioned on an electronic device of a first user to sense neuro-physiological responses of the first user and one or more second users. The one or more second users are in field-of-view (FOV) of the one or more sensors positioned on the electronic device of the first user. The first user and the one or more second users are participants of at least one of a social interaction application or an immersive content experience. The method may further include determining, based on the sensor data of the first user and the one or more second users, at least one of a composite neurological state (CNS) value for the social interaction application and a content engagement power (CEP) value for immersive content. The method may further include predicting, by a processor in conjunction with an artificial intelligence (AI) engine, one or more recommendations for one or more action items for the first user based on the sensor data of the first user and the one or more second users, and the at least one of the CNS value and the CEP value. The method may further include creating a feedback loop based on the sensor data of the first user and the one or more second users, the at least one of the CNS value and the CEP value, and the predicted one or more recommendations. Content of the feedback loop is rendered on an output unit of the electronic device of the first user during play of the at least one of the social interaction application and the immersive content. The progress of the at least one of a social interaction and the immersive content is controlled by the first user based on the predicted one or more recommendations rendered on the output unit of the electronic device of the first user.

In an aspect, the method may include monitoring at least one of a personal interaction the first user or audio-video content displayed on the output unit of the electronic device of the first user. The audio-video content is associated with digital data representing a social interaction of the first user or a user engagement of the first user with immersive content displayed at the output unit.

In an aspect, the at least one of the CNS value and the CEP value is determined using arousal values. The arousal values are based on the sensor data and comparison of a stimulation average arousal with an expectation average arousal. The stimulation average arousal is based on the sensor data.

In an aspect, the method may include detecting one or more stimulus events based on the sensor data exceeding a threshold value for a time period and determining the expectation average arousal based on sensor data measuring a like involuntary response of a user while engaged with known audio-video stimuli. The method may further include calculating one of multiple event powers for each of the first user and the one or more second users and for each of the stimulus events and aggregating the event powers. The method may further include assigning weights to each of the event powers based on one or more source identities for the sensor data. The method may further include calculating an expectation power for the known audio-video stimuli for the first user and the one or more second users and for each of the stimulus events.

The method may further include calculating the CNS value based on a ratio of a sum of the event powers to the expectation power for a comparable event in a social interaction. The method may further include calculating the CEP value based on a ratio of a sum of the event powers to the expectation power for a comparable event in corresponding genre.

The method may further include determining a digital representation of valence values based on the sensor data, and normalizing the digital representation of the valence values based on like values collected for a known audio-video stimuli.

In an aspect, the method may further include determining a valence error measurement based on comparing the digital representation of the valence values to a targeted valence value for at least one of a social interaction and a targeted emotional arc for the immersive content.

In an aspect, the social interaction application is one or more of a dating application or a social networking application. The controlled progress of the social interaction application includes at least one of: selecting a new challenge for the first user, matching the first user to one of the one or more second users, intervention by the first user upon viewing a report and overriding the match, or determining capabilities of an avatar associated with each of the first user and the one or more second users.

In an aspect, the immersive content is categorized as a spatial immersive content, strategic immersive content, narrative immersive content, or tactical immersive content. The immersive content is one of a card game, a bluffing game, an action video game, an adventure video game, a role-playing video game, a simulation video game, a strategy video game, a sports video game and a party video game. The controlled progress of the immersive content includes at least one of: determining a winner, changing a parameter setting for audio-visual game output, selecting a new challenge for the first user, or determining capabilities of an avatar associated with each of the first user and the one or more second users, or a non-player character.

The foregoing methods may be implemented in any suitable programmable computing apparatus, by provided program instructions in a non-transitory computer-readable medium that, when executed by a computer processor, cause the apparatus to perform the described operations. The processor may be local to the apparatus and user, located remotely, or may include a combination of local and remote processors. An apparatus may include a computer or set of connected computers that is used in measuring and communicating CNS or like engagement measures for content output devices. A content output device may include, for example, a personal computer, mobile phone, an audio receiver (e.g., a Bluetooth earpiece), notepad computer, a television or computer monitor, a projector, a virtual reality device, augmented reality device, or haptic feedback device. Other elements of the apparatus may include, for example, an audio output device and a user input device, which participate in the execution of the method. An apparatus may include a virtual or augmented reality device, such as a headset or other display that reacts to movements of a user's head and other body parts. The apparatus may include biometric sensors that provide data used by a controller to determine a digital representation of CNS.

To the accomplishment of the foregoing and related ends, one or more examples comprise the features hereinafter fully described and particularly pointed out in the claims. The following description and the annexed drawings set forth in detail certain illustrative aspects and are indicative of but a few of the various ways in which the principles of the examples may be employed. Other advantages and novel features will become apparent from the following detailed description when considered in conjunction with the drawings and the disclosed examples, which encompass all such aspects and their equivalents.

### BRIEF DESCRIPTION OF DRAWINGS

The features, nature, and advantages of the present disclosure will become more apparent from the detailed description set forth below when taken in conjunction with the drawings in which like reference characters identify like elements correspondingly throughout the specification and drawings.

FIG. 1 is a schematic block diagram illustrating aspects of a system and apparatus for digitally representing user engagement with audio-video content in a computer memory based on biometric sensor data, coupled to one or more distribution systems.

FIG. 2 is a schematic block diagram illustrating aspects of a server for digitally representing user engagement with audio-video content in a computer memory based on biometric sensor data.

FIG. 3 is a schematic block diagram illustrating aspects of a client device for digitally representing user engagement with audio-video content in a computer memory based on biometric sensor data.

FIG. 4 is a schematic diagram showing features of a virtual-reality client device for digitally representing user engagement with audio-video content in a computer memory based on biometric sensor data.

FIG. 5 is a flow chart illustrating high-level operation of a method determining a digital representation of CNS based on biometric sensor data collected during performance of a video game or other application facilitating social interaction.

FIG. 6 is a block diagram illustrating high-level aspects of a system for digitally representing user engagement with audio-video content in a computer memory based on biometric sensor data.

FIG. 7A is a diagram indicating an arrangement of neuro-physiological states relative to axes of a two-dimensional neuro-physiological space.

FIG. 7B is a diagram indicating an arrangement of neuro-physiological states relative to axes of a three-dimensional neuro-physiological space.

FIG. 8 is a flow chart illustrating a process and algorithms for determining a content engagement rating based on biometric response data.

FIG. 9 is a perspective view of a user using a mobile application with sensors and accessories for collecting biometric data used in the methods and apparatus described herein.

FIG. 10 is a flow chart illustrating aspects of a method for controlling a social interaction application using biometric sensor data.

FIG. 11 is a flow chart illustrating measurement of neuro-physiological state in a player or user.

FIG. 12 is a diagram illustrating a system including mobile devices with biometric sensors to enhance interpersonal communication with biometric tells.

FIG. 13 is a flow chart illustrating aspects of a method for operating a system for enhancing interpersonal communication with biometric tells.

FIGS. 14-16 are flow charts illustrating optional further aspects or operations of the method diagrammed in FIG. 13.

FIG. 17 is a conceptual block diagram illustrating components of an apparatus or system for enhancing interpersonal communication with biometric tells.

FIG. 18 shows aspects of a method or methods for controlling progress of social interaction or immersive content based on sensor data of multiple users, composite neuro-physiological state and/or content engagement power.

FIG. 19 shows a system corresponding to a social interaction between two users.

FIG. 20 shows a system corresponding to viewing an immersive content by a user.

FIG. 21 is a conceptual block diagram illustrating components of an apparatus or system for controlling progress of audio-video content based on sensor data of multiple users, composite neuro-physiological state and/or content engagement power.

### DETAILED DESCRIPTION

Various aspects are now described with reference to the drawings. In the following description, for purposes of explanation, numerous specific details are set forth in order to provide a thorough understanding of one or more aspects. It may be evident, however, that the various aspects may be practiced without these specific details. In other instances, well-known structures and devices are shown in block diagram form to facilitate describing these aspects.

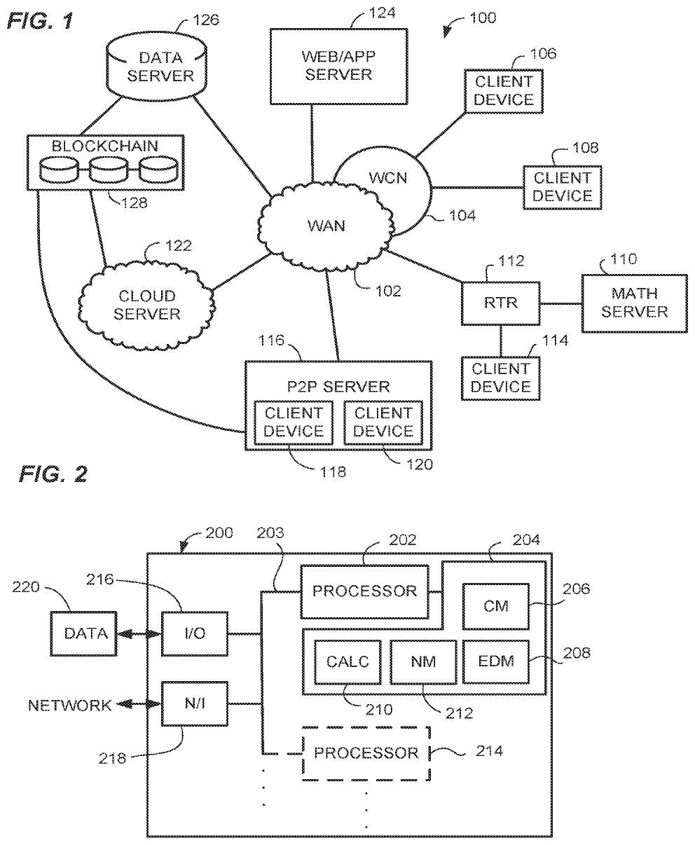

Referring to FIG. 1, methods for signal processing of biometric sensor data for detection of neuro-physiological state in communication enhancement applications may be implemented in a client-server environment 100. Other architectures may also be suitable. In a network architecture, sensor data can be collected and processed locally, and transmitted to a server that processes biometric sensor data from one or more subjects, calculating a digital representation of user neuro-physiological state based on biometric sensor data in a computer memory and using the digital representation to control a machine, to control a communication or game process, or to inform a user of the digital representation of the user's own neuro-physiological state or the state of another user in a personal communication or game.

A suitable client-server environment 100 may include various computer servers and client entities in communication via one or more networks, for example a Wide Area Network (WAN) 102 (e.g., the Internet) and/or a wireless communication network (WCN) 104, for example a cellular telephone network. Computer servers may be implemented in various architectures. For example, the environment 100 may include one or more Web/application servers 124 containing documents and application code compatible with World Wide Web protocols, including but not limited to HTML, XML, PHP and Javascript documents or executable scripts, for example. The Web/application servers 124 may serve applications for outputting a video game or other application facilitating social interaction and for collecting biometric sensor data from users experiencing the content. In an alternative, data collection applications may be served from a math server 110, a cloud-based data server 122, blockchain data structure 128, or content data server 126. As described in more detail herein below, the environment for experiencing a video game or other application facilitating social interaction may include a physical set for live interactive theater, or a combination of one or more data collection clients feeding data to a modeling and rendering engine that serves a virtual theater.

The environment 100 may include one or more data servers 126 for holding data, for example video, audio-video, audio, and graphical content components of game or social media application content for consumption using a client device, software for execution on or in conjunction with client devices, for example sensor control and sensor signal processing applications, and data collected from users or client devices. Data collected from client devices or users may include, for example, sensor data and application data. Sensor data may be collected by a background (not user-facing) application operating on the client device, and transmitted to a data sink, for example, the cloud-based data server 122 or discrete data server 126. Application data means application state data, including but not limited to records of user interactions with an application or other application inputs, outputs or internal states. Applications may include software for video games, social interaction, or personal training. Applications and data may be served from other types of servers, for example, any server accessing a distributed blockchain data structure 128, or a peer-to-peer (P2P) server 116 such as may be provided by a set of client devices 118, 120 operating contemporaneously as micro-servers or clients.

As used herein, “users” are always consumers of video games or social interaction applications from which a system node collects neuro-physiological response data for use in determining a digital representation of emotional state for use in the game or other social interaction. When actively participating in a game or social experience via an avatar or other agency, users may also be referred to herein as player actors. Viewers are not always users. For example, a bystander may be a passive viewer from which the system collects no biometric response data. As used herein, a “node” includes a client or server participating in a computer network.

The network environment 100 may include various client devices, for example a mobile smart phone client 106 and notepad client 108 connecting to servers via the WCN 104 and WAN 102 or a mixed reality (e.g., virtual reality or augmented reality) client device 114 connecting to servers via a router 112 and the WAN 102. In general, client devices may be, or may include, computers used by users to access video games or other applications facilitating social interaction provided via a server or from local storage. In an aspect, the data processing server 110 may determine digital representations of biometric data for use in real-time or offline applications. Real-time applications may include, for example, video games, in-person social games with emotional feedback via a client device, applications for personal training and self-improvement, and applications for live social interaction, e.g., text chat, voice chat, video conferencing, and virtual presence conferencing. Offline applications may include, for example, ‘green lighting’ production proposals, automated screening of production proposals prior to green lighting, automated or semi-automated packaging of promotional content such as trailers or video ads, and customized editing or design of content for targeted users or user cohorts (both automated and semi-automated).

FIG. 2 shows a data processing server 200 for digitally representing user engagement with a video game or other application facilitating social interaction in a computer memory based on biometric sensor data, which may operate in the environment 100, in similar networks, or as an independent server. The server 200 may include one or more hardware processors 202, 214 (two of one or more shown). Hardware includes firmware. Each of the one or more processors 202, 214 may be coupled to an input/output port 216 (for example, a Universal Serial Bus port or other serial or parallel port) to a source 220 for biometric sensor data indicative of users' neuro-physiological states. Some types of servers, e.g., cloud servers, server farms, or P2P servers, may include multiple instances of discrete servers 200 that cooperate to perform functions of a single server.

The server 200 may include a network interface 218 for sending and receiving applications and data, including but not limited to sensor and application data used for digitally representing user neuro-physiological state during a game or social interaction in a computer memory based on biometric sensor data. The content may be served from the server 200 to a client device or stored locally by the client device. If stored local to the client device, the client and server 200 may cooperate to handle collection of sensor data and transmission to the server 200 for processing.

Each processor 202, 214 of the server 200 may be operatively coupled to at least one memory 204 holding functional modules 206, 208, 210, 212 of an application or applications for performing a method as described herein. The modules may include, for example, a correlation module 206 that correlates biometric feedback to one or more metrics such as arousal or valence. The correlation module 206 may include instructions that when executed by the processor 202 and/or 214 cause the server to correlate biometric sensor data to one or more neuro-physiological (e.g., emotional) states of the user, using machine learning (ML) or other processes. An event detection module 208 may include functions for detecting events based on a measure or indicator of one or more biometric sensor inputs exceeding a data threshold. The modules may further include, for example, a normalization module 210. The normalization module 210 may include instructions that when executed by the processor 202 and/or 214 cause the server to normalize measures of valence, arousal, or other values using a baseline input. The modules may further include a calculation function 212 that when executed by the processor causes the server to calculate a Composite Neuro-physiological State (CNS) based on the sensor data and other output from upstream modules. Details of determining a CNS are disclosed later herein. The memory 204 may contain additional instructions, for example an operating system, and supporting modules.

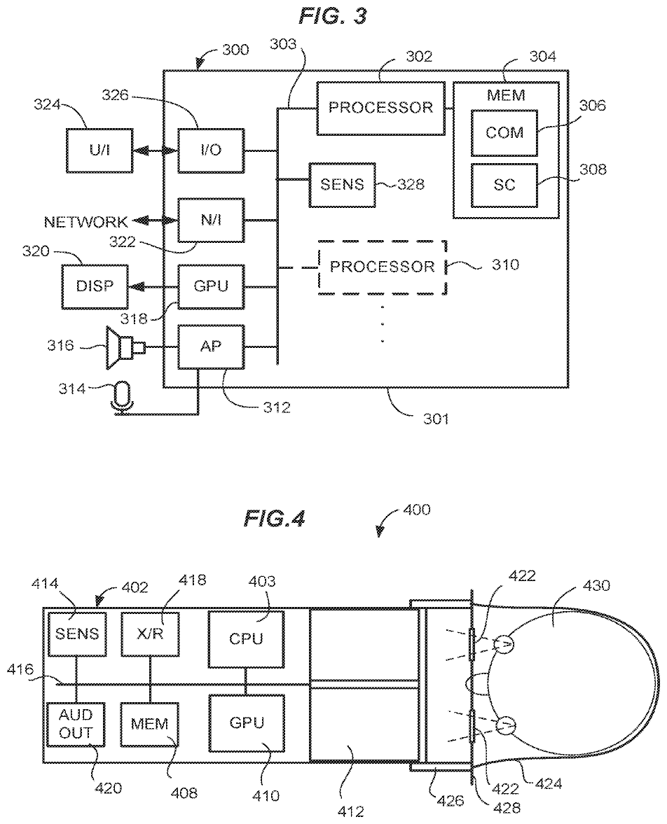

Referring to FIG. 3, a content consumption device 300 generates biometric sensor data indicative of a user's neuro-physiological response to output generated from a video game or other application facilitating social interaction signaling. The content consumption device 300 may include, for example, a processor 302, for example a central processing unit based on 80×86 architecture as designed by Intel™ or AMD™, a system-on-a-chip as designed by ARM™, or any other suitable microprocessor. The processor 302 may be communicatively coupled to auxiliary devices or modules of the 3D environment apparatus, such as the content consumption device 300, using a bus or other coupling. Optionally, the processor 302 and its coupled auxiliary devices or modules may be housed within or coupled to a housing 301, for example, a housing having a form factor of a television, set-top box, smartphone, wearable googles, glasses, or visor, or other form factor.

A user interface device 324 may be coupled to the processor 302 for providing user control input to a media player and data collection process. The process may include outputting video and audio for a display screen or projection display device. In some embodiments, a video game or other application facilitating social interaction control process may be, or may include, audio-video output for an immersive mixed reality content display process operated by a mixed reality immersive display engine executing on the processor 302.

User control input may include, for example, selections from a graphical user interface or other input (e.g., textual or directional commands) generated via a touch screen, keyboard, pointing device (e.g., game controller), microphone, motion sensor, camera, or some combination of these or other input devices represented by user interface device 324. Such user interface device 324 may be coupled to the processor 302 via an input/output port 326, for example, a Universal Serial Bus (USB) or equivalent port. Control input may also be provided via a sensor 328 coupled to the processor 302. A sensor 328 may be or may include, for example, a motion sensor (e.g., an accelerometer), a position sensor, a camera or camera array (e.g., stereoscopic array), a biometric temperature or pulse sensor, a touch (pressure) sensor, an altimeter, a location sensor (for example, a Global Positioning System (GPS) receiver and controller), a proximity sensor, a motion sensor, a smoke or vapor detector, a gyroscopic position sensor, a radio receiver, a multi-camera tracking sensor/controller, an eye-tracking sensor, a microphone or a microphone array, an electroencephalographic (EEG) sensor, a galvanic skin response (GSR) sensor, a facial electromyography (fEMG) sensor, an electrocardiogram (EKG) sensor, a video facial action unit (FAU) sensor, a brain machine interface (BMI) sensor, a video pulse detection (VPD) sensor, a pupil dilation sensor, a body chemical sensor, a functional magnetic resonance imaging (fMRI) sensor, a photoplethysmography (PPG) sensor, phased-array radar (PAR) sensor, or a functional near-infrared data (fNIR) sensor. Any one or more of an eye-tracking sensor, FAU sensor, PAR sensor, pupil dilation sensor or heartrate sensor may be or may include, for example, a front-facing (or rear-facing) stereoscopic camera such as used in the iPhone 10 and other smartphones for facial recognition. Likewise, cameras in a smartphone or similar device may be used for ambient light detection, for example, to detect ambient light changes for correlating to changes in pupil dilation.

The sensor or sensors 328 may detect biometric data used as an indicator of the user's neuro-physiological state, for example, one or more of facial expression, skin temperature, pupil dilation, respiration rate, muscle tension, nervous system activity, pulse, EEG data, GSR data, fEMG data, EKG data, FAU data, BMI data, pupil dilation data, chemical detection (e.g., oxytocin) data, fMRI data, PPG data or fNIR data. In addition, the sensor(s) 328 may detect a user's context, for example an identity position, size, orientation and movement of the user's physical environment and of objects in the environment, motion or other state of a user interface display, for example, motion of a virtual-reality headset. Sensors may be built into wearable gear or may be non-wearable, including a display device, or in auxiliary equipment such as a smart phone, smart watch, or implanted medical monitoring device. Sensors may also be placed in nearby devices such as, for example, an Internet-connected microphone and/or camera array device used for hands-free network access or in an array over a physical set.

Sensor data from the one or more sensors 328 may be processed locally by the processor 302 to control display output, and/or transmitted to a server 200 for processing by the server in real time, or for non-real-time processing. As used herein, “real time” refers to processing responsive to user input without any arbitrary delay between inputs and outputs; that is, that reacts as soon as technically feasible. “Non-real time” or “offline” refers to batch processing or other use of sensor data that is not used to provide immediate control input for controlling the display, but that may control the display after some arbitrary amount of delay.

To enable communication with another node of a computer network, for example a video game or other application facilitating social interaction server 200, the client device 300 may include a network interface 322, e.g., an Ethernet port, wired or wireless. Network communication may be used, for example, to enable multiplayer experiences, including immersive or non-immersive experiences of a video game or other application facilitating social interaction such as non-directed multi-user applications, for example social networking, group entertainment experiences, instructional environments, video gaming, and so forth. Network communication can also be used for data transfer between the client and other nodes of the network, for purposes including data processing, content delivery, content control, and tracking. The client may manage communications with other network nodes using a communications module 306 that handles application-level communication needs and lower-level communications protocols, preferably without requiring user management.

A display device 320 may be coupled to the processor 302, for example via a graphics processing unit 318 integrated in the processor 302 or in a separate chip. The display device 320 may include, for example, a flat screen color liquid crystal display (LCD) illuminated by light-emitting diodes (LEDs) or other lamps, a projector driven by an LCD or by a digital light processing (DLP) unit, a laser projector, or other digital display device. The display device 320 may be incorporated into a virtual reality headset or other immersive display system, or may be a computer monitor, home theater or television screen, or projector in a screening room or theater. In a real social interaction application, clients for users and actors may avoid using a display in favor of audible input through an earpiece or the like, or tactile impressions through a tactile suit.

In virtual social interaction applications, video output driven by a mixed reality display engine operating on the processor 302, or other application for coordinating user inputs with an immersive content display and/or generating the display, may be provided to the display device 320 and output as a video display to the user. Similarly, an amplifier/speaker or other audio output transducer 316 may be coupled to the processor 302 via an audio processor 312. Audio output correlated to the video output and generated by the media player module 308, a video game or other application facilitating social interaction or other application may be provided to the audio transducer 316 and output as audible sound to the user. The audio processor 312 may receive an analog audio signal from a microphone 314 and convert it to a digital signal for processing by the processor 302. The microphone can be used as a sensor for detection of neuro-physiological (e.g., emotional) state and as a device for user input of verbal commands, or for social verbal responses to other users.

The 3D environment apparatus, such as the content consumption device 300, may further include a random-access memory (RAM) 304 holding program instructions and data for rapid execution or processing by the processor during controlling of a video game or other application facilitating social interaction in response to biosensor data collected from a user. When the client device 300 is powered off or in an inactive state, program instructions and data may be stored in a long-term memory, for example, a non-volatile magnetic, optical, or electronic memory storage device (not shown). Either or both RAM 304 or the storage device may comprise a non-transitory computer-readable medium holding program instructions, that when executed by the processor 302, cause the client device 300 to perform a method or operations as described herein. Program instructions may be written in any suitable high-level language, for example, C, C++, C #, JavaScript, PHP, or Java™, and compiled to produce machine-language code for execution by the processor.

Program instructions may be grouped into functional modules 306, 308, to facilitate coding efficiency and comprehensibility. A communication module 306 may include coordinating communication of biometric sensor data or metadata to a calculation server. A sensor control module 308 may include controlling sensor operation and processing raw sensor data for transmission to a calculation server. The modules 306, 308, even if discernable as divisions or grouping in source code, are not necessarily distinguishable as separate code blocks in machine-level coding. Code bundles directed toward a specific type of function may be considered to comprise a module, regardless of whether or not machine code on the bundle can be executed independently of other machine code. The modules may be high-level modules only. The media player module 308 may perform operations of any method described herein, and equivalent methods, in whole or in part. Operations may be performed independently or in cooperation with another network node or nodes, for example, the server 200.

The content control methods disclosed herein may be used with Virtual Reality (VR) or Augmented Reality (AR) output devices, for example in virtual live or robotic interactive theater. FIG. 4 is a schematic diagram illustrating one type of immersive VR stereoscopic display device 400, as an example of the client device 300 in a more specific form factor. The client device 300 may be provided in various form factors, of which immersive VR stereoscopic display device 400 provides but one example. The innovative methods, apparatus and systems described herein are not limited to a single form factor, but may be used with any output device suitable for communicating a representation of a user's CNS to a person. As used herein, “a social interaction application signal” includes any digital signal from a video game or other application facilitating social interaction. In an aspect, the operation of the video game or other application facilitating social interaction may vary in response to a detected neuro-physiological state of the user calculated from biometric sensor data. In virtual reality or augmented reality applications, the appearance, behavior, and capabilities of a user's avatar may be controlled in response to the user's CNS, providing greater realism, interest, or enjoyment of the game play or social experience.

Whether in an immersive environment or non-immersive environment, the application may control the appearance, behavior, and capabilities of a computer-controlled non-player character in response to real-time CNS data from one or more users. For example, if CNS data indicates low arousal, a controller may increase difficulty or pace of the experience, may modify characteristics of avatars, non-player characters, the playing environment, or a combination of the foregoing. For further example, if CNS data indicates excessive tension or frustration, the controller may similarly reduce difficulty or pace of the experience.

The immersive VR stereoscopic display device 400 may include a tablet support structure made of an opaque lightweight structural material (e.g., a rigid polymer, aluminum or cardboard) configured for supporting and allowing for removable placement of a portable tablet computing or smartphone device including a high-resolution display screen, for example, an LCD. The immersive VR stereoscopic display device 400 is designed to be worn close to the user's face, enabling a wide field of view using a small screen size such as in a smartphone. The support structure 426 holds a pair of lenses 422 in relation to the display screen 412. The lenses may be configured to enable the user to comfortably focus on the display screen 412 which may be held approximately one to three inches from the user's eyes.

The immersive VR stereoscopic display device 400 may further include a viewing shroud (not shown) coupled to the support structure 426 and configured of a soft, flexible or other suitable opaque material for form fitting to the user's face and blocking outside light. The shroud may be configured to ensure that the only visible light source to the user is the display screen 412, enhancing the immersive effect of using the immersive VR stereoscopic display device 400. A screen divider may be used to separate the display screen 412 into independently driven stereoscopic regions, each of which is visible only through a corresponding one of the lenses 422. Hence, the immersive VR stereoscopic display device 400 may be used to provide stereoscopic display output, providing a more realistic perception of 3D space for the user.

The immersive VR stereoscopic display device 400 may further comprise a bridge (not shown) for positioning over the user's nose, to facilitate accurate positioning of the lenses 422 with respect to the user's eyes. The immersive VR stereoscopic display device 400 may further comprise an elastic strap or band, or other headwear for fitting around the user's head and holding the immersive VR stereoscopic display device 400 to the user's head.

The immersive VR stereoscopic display device 400 may include additional electronic components of a display and communications unit 402 (e.g., a tablet computer or smartphone) in relation to a user's head 430. When wearing the support structure 426, the user views the display screen 412 though the pair of lenses 422. The display screen 412 may be driven by the Central Processing Unit (CPU) 403 and/or Graphics Processing Unit (GPU) 410 via an internal bus 417. Components of the display and communications unit 402 may further include, for example, a transmit/receive component or components 418, enabling wireless communication between the CPU and an external server via a wireless coupling. The transmit/receive component 418 may operate using any suitable high-bandwidth wireless technology or protocol, including, for example, cellular telephone technologies such as 3rd 4th or 5th Generation Partnership Project (3GPP) Long Term Evolution (LTE) also known as 3G, 4G, or 5G, Global System for Mobile communications (GSM) or Universal Mobile Telecommunications System (UMTS), and/or a wireless local area network (WLAN) technology for example using a protocol such as Institute of Electrical and Electronics Engineers (IEEE) 802.11. The transmit/receive component or components 418 may enable streaming of video data to the display and communications unit 402 from a local or remote video server, and uplink transmission of sensor and other data to the local or remote video server for control or audience response techniques as described herein.

Components of the display and communications unit 402 may further include, for example, one or more sensors 414 coupled to the CPU 403 via the communications bus 417. Such sensors may include, for example, an accelerometer/inclinometer array providing orientation data for indicating an orientation of the display and communications unit 402. As the display and communications unit 402 is fixed to the user's head 430, this data may also be calibrated to indicate an orientation of the head 430. The one or more sensors 414 may further include, for example, a Global Positioning System (GPS) sensor indicating a geographic position of the user. The one or more sensors 414 may further include, for example, a camera or image sensor positioned to detect an orientation of one or more of the user's eyes, or to capture video images of the user's physical environment (for VR mixed reality), or both. In some embodiments, a camera, image sensor, or other sensor configured to detect a user's eyes or eye movements may be mounted in the support structure 426 and coupled to the CPU 403 via the bus 416 and a serial bus port (not shown), for example, a Universal Serial Bus (USB) or other suitable communications port. The one or more sensors 414 may further include, for example, an interferometer positioned in the support structure 404 and configured to indicate a surface contour to the user's eyes. The one or more sensors 414 may further include, for example, a microphone, an array of microphones, or other audio input transducer for detecting spoken user commands or verbal and non-verbal audible reactions to display output. The one or more sensors may include a subvocalization mask using electrodes as described by Arnav Kapur, Pattie Maes and Shreyas Kapur in a paper presented at the Association for Computing Machinery's ACM Intelligent User Interface conference in 2018. Subvocalized words might be used as command input, as indications of arousal or valence, or both. The one or more sensors may include, for example, electrodes or microphone to sense heart rate, a temperature sensor configured for sensing skin or body temperature of the user, an image sensor coupled to an analysis module to detect facial expression or pupil dilation, a microphone to detect verbal and nonverbal utterances, or other biometric sensors for collecting biofeedback data including nervous system responses capable of indicating emotion via algorithmic processing, including any sensor as already described in connection with FIG. 3 at 328.

Components of the display and communications unit 402 may further include, for example, an audio output transducer 420, for example a speaker or piezoelectric transducer in the display and communications unit 402 or audio output port for headphones or other audio output transducer mounted in headgear 424 or the like. The audio output device may provide surround sound, multichannel audio, so-called ‘object-oriented audio’, or other audio track output accompanying stereoscopic immersive VR video display content. Components of the display and communications unit 402 may further include, for example, a memory 408 coupled to the CPU 403 via a memory bus. The memory 408 may store, for example, program instructions that when executed by the processor cause the immersive VR stereoscopic display device 400 to perform operations as described herein. The memory 408 may also store data, for example, audio-video data in a library or buffered during streaming from a network node.

Having described examples of suitable clients, servers, and networks for performing signal processing of biometric sensor data for detection of neuro-physiological state in communication enhancement applications, more detailed aspects of suitable signal processing methods will be addressed. FIG. 5 illustrates an overview of a method 500 for calculating a Composite Neuro-physiological State (CNS), which may include four related operations in any functional order or in parallel. The operations may be programmed into executable instructions for a server as described herein.

A correlating operation 510 uses an algorithm to correlate biometric data for a user or user cohort to a neuro-physiological indicator. Optionally, the algorithm may be a machine-learning algorithm configured to process context-indicating data in addition to biometric data, which may improve accuracy. Context-indicating data may include, for example, user location, user position, time-of-day, day-of-week, ambient light level, ambient noise level, and so forth. For example, if the user's context is full of distractions, biofeedback data may have a different significance from that in a quiet environment.

As used herein, a “neuro-physiological indicator” is a machine-readable symbolic value that relates to a real-time neuro-physiological state of a user engaged in a social interaction. The indicator may have constituent elements, which may be quantitative or non-quantitative. For example, an indicator may be designed as a multi-dimensional vector with values representing intensity of psychological qualities such as cognitive load, arousal, and valence. “Valence” in psychology and as used herein means the state of attractiveness or desirability of an event, object or situation; valence is said to be positive when a subject feels something is good or attractive and negative when the subject feels the object is repellant or bad. “Arousal” in psychology and as used herein means the state of alertness and attentiveness of the subject. A machine learning algorithm may include at least one supervised machine learning (SML) algorithm, for example, one or more of a linear regression algorithm, a neural network algorithm, a support vector algorithm, a naive Bayes algorithm, a linear classification module or a random forest algorithm.

An event detection operation 520 analyzes a time-correlated signal from one or more sensors during output of a video game or other application facilitating social interaction to a user and detects events wherein the signal exceeds a threshold. The threshold may be a fixed predetermined value, or a variable number such as a rolling average. An example for GSR (galvanic skin response) data is provided herein below. Discrete measures of neuro-physiological response may be quantified for each event. Neuro-physiological state cannot be measured directly therefore sensor data indicates sentic modulation. Sentic modulations are modulations of biometric waveforms attributed to neuro-physiological states or changes in neuro-physiological states. In an aspect, to obtain baseline correlations between sentic modulations and neuro-physiological states, player actors may be shown a known visual stimulus (e.g., from focus group testing or a personal calibration session) to elicit a certain type of emotion. While under the stimulus, the test module may capture the player actor's biometric data and compare stimulus biometric data to resting biometric data to identify sentic modulation in biometric data waveforms.

CNS measurement and related methods may be used as a driver or control parameter for social interaction applications. Measured errors between intended effects and group response may be useful for informing design of a video game or other application facilitating social interaction, distribution and marketing, or any activity that is influenced by a cohort's neuro-physiological response to a social interaction application. In addition, the measured errors can be used in a computer-implemented application module to control or influence real-time operation of a social interaction application experience. Use of smartphones or tablets may be useful during focus group testing because such programmable devices already include one or more sensors for collection of biometric data. For example, Apple's™ iPhone™ includes front-facing stereographic cameras that may be useful for eye tracking, FAU detection, pupil dilation measurement, heartrate measurement and ambient light tracking, for example. Participants in the focus group may view the social interaction application on the smartphone or similar device, which collects biometric data with the participant's permission by a focus group application operating on their viewing device.

A normalization operation 530 performs an arithmetic or other numeric comparison between test data for known stimuli and the measured signal for the user and normalizes the measured value for the event. Normalization compensates for variation in individual responses and provides a more useful output. Once the input sensor events are detected and normalized, a calculation operation 540 determines a CNS value for a user or user cohort and records the values in a time-correlated record in a computer memory.

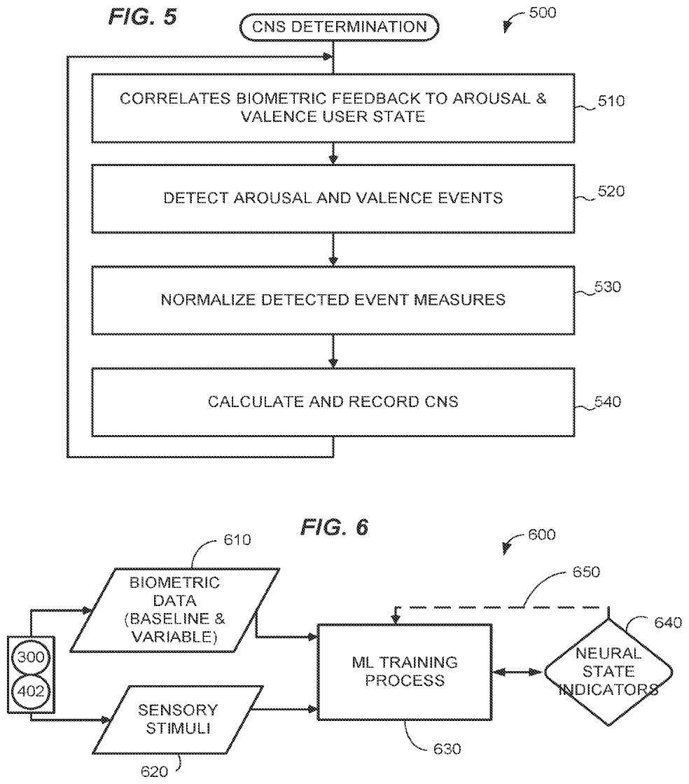

Machine learning, also called AI, can be an efficient tool for uncovering correlations between complex phenomena. As shown in FIG. 6, a system 600 responsive to sensor data 610 indicating a user's neuro-physiological state may use a machine learning training process 630 to detect correlations between sensory stimuli data 620 from a social interaction application experience and biometric data 610. The training process 630 may receive stimuli data 620 that is time-correlated to the biometric data 610 from media player clients (e.g., clients 300, 402). The data may be associated with a specific user or cohort, or may be generic. Both types of input data (associated with a user and generic) may be used together. Generic input data can be used to calibrate a baseline for neuro-physiological response to a scene, to classify a baseline neuro-physiological response to stimuli that simulates social interaction. For example, if most users exhibit similar biometric tells when engaged with similar social interactions (e.g., friendly, happy, angry, scary, seductive, etc.), each similar interaction can be classified with like interactions that provoke similar biometric data from users. As used herein, biometric data provides a “tell” on how a user thinks and feels about their experience of a video game or other application facilitating social interaction, i.e., the user's neuro-physiological response to the game or social interaction. The similar interactions may be collected and reviewed by a human, who may score the interactions on neuro-physiological indicator metrics 640 using automated analysis tools. In an alternative, the indicator metrics 640 can be scored by human and semi-automatic processing without being classed with similar interactions. Human-scored elements of the social interaction application production can become training data for the machine learning training process 630. In some embodiments, humans scoring elements of a video game or other application facilitating social interaction may include the users, such as via online survey forms. Scoring should consider cultural demographics and may be informed by expert information about responses of different cultures to scene elements.

The ML training process 630 compares human and machine-determined scores of social interactions and uses iterative machine learning methods as known in the art to reduce error between the training data and its own estimates. Creative content analysts may score data from multiple users based on their professional judgment and experience. Individual users may score their own social interactions. For example, users willing to assist in training their personal ‘director software’ to recognize their neuro-physiological states might score their own emotions while playing a game or engaging in other social interaction. A problem with this approach is that the user scoring may interfere with their normal reactions, misleading the machine learning algorithm. Other training approaches include clinical testing of subject biometric responses over short social interactions, followed by surveying the clinical subjects regarding their neuro-physiological states. A combination of these and other approaches may be used to develop training data for the machine learning training process 630.

Composite Neuro-physiological State is a measure of composite neuro-physiological response throughout the user experience of a video game or other application facilitating social interaction, which may be monitored and scored during or after completion of the experience for different time periods. Overall user enjoyment is measured as the difference between expectation biometric data modulation power (as measured during calibration) and the average sustained biometric data modulation power. Measures of user engagement may be made by other methods and correlated to Composite Neuro-physiological State or made a part of scoring Composite Neuro-physiological State. For example, exit interview responses or acceptance of offers to purchase, subscribe, or follow may be included in or used to tune calculation of Composite Neuro-physiological State. Offer-response rates may be used during or after participation in a social interaction experience to provide a more complete measure of user neuro-physiological response. However, it should be appreciated that the purpose of calculating CNS does not necessarily include increasing user engagement with passive content, but may be primarily directed to controlling aspects of game play for providing a different and more engaging user experience of social interactions.

The user's mood going into the interaction affects how the narrative entertainment is interpreted so the computation of CNS might calibrate mood out. If a process is unable to calibrate out mood, then it may take it into account in the operation of the social media application. For example, if a user's mood is depressed, a social interaction application might favor more positively valenced interactions or matching to more sympathetic partners. For further example, if a user's mood is elevated, the application might favor more challenging encounters. The instant systems and methods of the present disclosure will work best for healthy and calm individuals though it will enable use of CNS in controlling operation of social interaction applications for everyone who partakes.

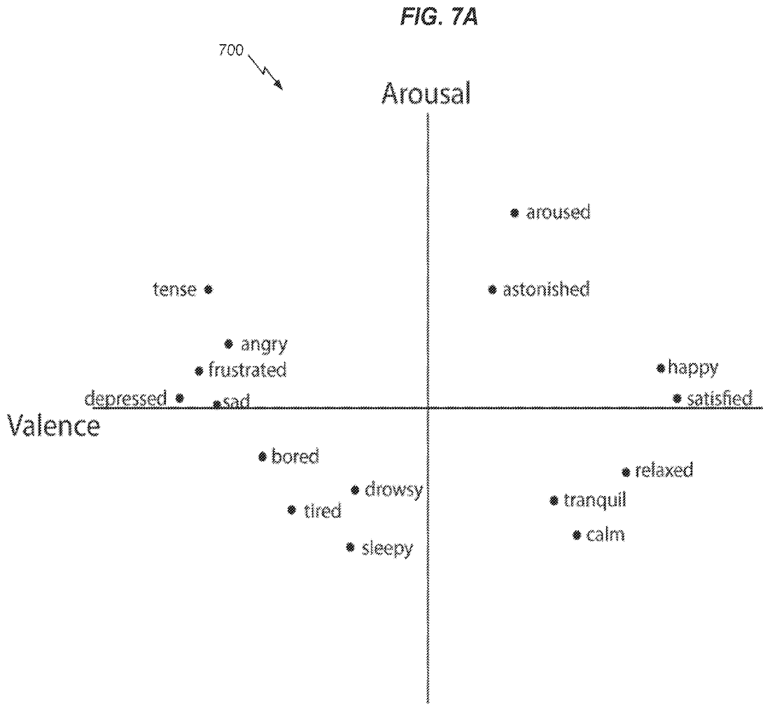

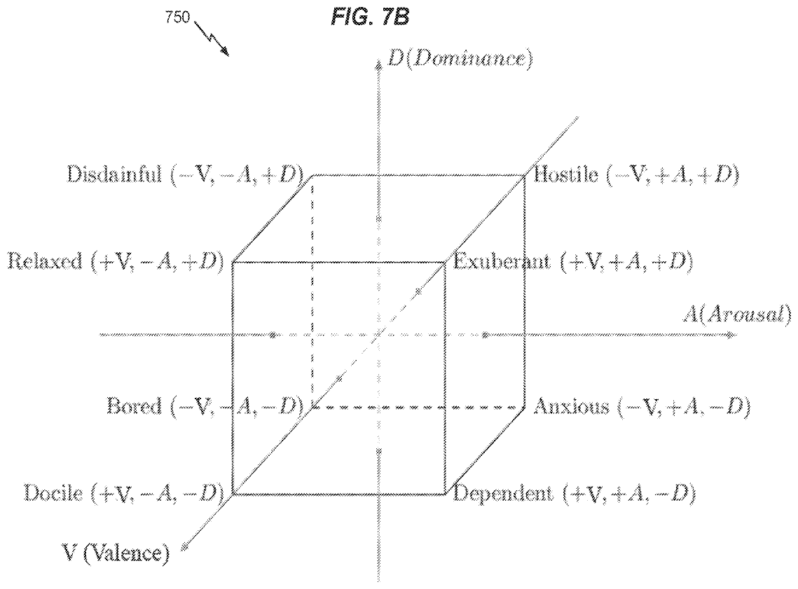

FIG. 7A shows an arrangement 700 of neuro-physiological states relative to axes of a two-dimensional neuro-physiological space defined by a horizontal valence axis and a vertical axis arousal. The illustrated emotions based on a valence/arousal neuro-physiological model are shown in the arrangement merely as an example, not actual or typical measured values. A media player client may measure valence with biometric sensors that measure facial action units, while arousal measurements may be done via GSR measurements for example.

Neuro-physiological spaces may be characterized by more than two axes. FIG. 7B diagrams a three-dimensional cognitive appraisal model 750 of a neuro-physiological space, wherein the third axis is social dominance or confidence. The model 750 illustrates a WAD′ (valence, arousal, dominance) model. The 3D model 1550 may be useful for complex emotions where a social hierarchy is involved. In another embodiment, a neuro-physiological state measure from biometric data may be modeled as a three-dimensional vector which provides cognitive workload, arousal and valence from which a processor can determine primary and secondary emotions after calibration. Engagement measures may be generalized to an N-dimensional model space wherein N is one or greater. In examples described herein, CNS is in a two-dimensional space, as shown in the arrangement 700, with valence and arousal axes, but CNS is not limited thereby. For example, dominance is another psychological axis of measurement that might be added, other axes may be added, and base axes other than valence and arousal might also be useful. Baseline arousal and valence may be determined on an individual basis during emotion calibration.

In the following detailed example, neuro-physiological state determination from biometric sensors is based on the valence/arousal neuro-physiological model where valence is positive/negative and arousal is magnitude. From this model, producers of social interaction application and other creative productions can verify the intention of the social experience by measuring social theory constructs such as tension (hope vs. fear) and rising tension (increase in arousal over time) and more. During social interaction mediated through the application, an algorithm can use the neuro-physiological model for operation of the application dynamically based on the psychological state or predisposition of the user. The inventive concepts described herein are not limited to the CNS neuro-physiological model described herein and may be adapted for use with any useful neuro-physiological model characterized by quantifiable parameters.

In a test environment, electrodes and other sensors can be placed manually on subject users in a clinical function. For consumer applications, sensor placement should be less intrusive and more convenient. For example, image sensors in visible and infrared wavelengths can be built into display equipment. For further example, a phased-array radar emitter may be fabricated as a microdevice and placed behind the display screen of a mobile phone or tablet, for detecting biometric data such as Facial Action Units or pupil dilation. Where a user wears gear or grasps a controller as when using VR equipment, electrodes can be built into headgear, controllers, and other wearable gear to measure skin conductivity, pulse, and electrical activity.

Target story arcs based on a video game or other application facilitating social interaction can be stored in a computer database as a sequence of targeted values in any useful neuro-physiological model for representing user neuro-physiological state in a social interaction, for example a valence/arousal model. Using the example of a valence/arousal model, a server may perform a difference calculation to determine the error between the planned/predicted and measured arousal and valence. The error may be used in application control or for generating an easily understood representation. Once a delta between the predicted and measured values passes a threshold, then the social interaction application software may command a branching action. For example, if the user's valence is in the ‘wrong’ direction based on the game design then the processor may change the content by the following logic: If absolute value of (Valence Predict-Valence Measured)>0 then Change Content. The change in content can be several different items specific to what the software has learned about the player-actor or it can be a trial or recommendation from an AI process. Likewise, if the arousal error falls below a threshold (e.g. 50%) of predicted (Absolute value of (error)>0.50*Predict) then the processor may change the content.

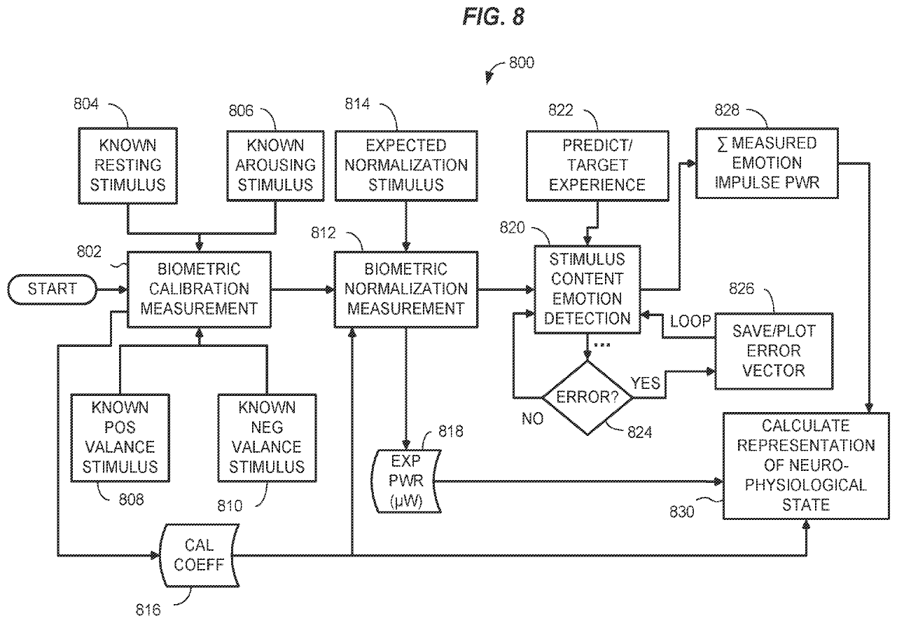

FIG. 8 shows a method 800 for determining a content rating for a video game or other application facilitating social interaction, including Composite Neuro-physiological State (CNS). The method may be implemented by encoding as an algorithm executable by a computer processor and applied in other methods described herein wherever a calculation of CNS is called for. CNS may be expressed as a ratio of a sum of event power ‘Pᵥ’ for the subject content to expectation power ‘Pₓ’ for a comparable event in a social interaction. Pᵥ and Pₓ are calculated using the same methodology for different subject matter and in the general case for different users. As such, the sums cover different total times, event power Pᵥ covering a time period ‘tᵥ’ that equals a sum of ‘n’ number of event power periods Δtᵥ for the subject content:

tᵥ=Σₙ¹Δtᵥ Eq. 1

Likewise, expectation power Pₓ covers a period ‘tₓ’ that equals a sum of ‘m’ number of event power periods Δtₓ for the expectation content:

tₓ=Σₘ¹Δtₓ Eq. 2

Each of powers Pᵥ and Pₓ is, for any given event ‘n’ or ‘m’, a dot product of a power vector P and a weighting vector W of dimension i, as follows:

Pᵥ_{ₙ}= [symbol] · [symbol] =Σᵢ¹Pᵥ_{ᵢ}Wᵢ=Pᵥ_{₁}W₁+Pᵥ_{₂}W₂+ . . . +Pᵥ_{ᵢ}Wᵢ Eq. 3

Pₓ_{ₘ}= [symbol] · [symbol] =Σᵢ¹PₓWᵢ=Pₓ_{₁}W₁+Pₓ_{₂}W₂+ . . . +Pₓ_{ᵢ}Wᵢ Eq. 4

In general, the power vector \vec{P} can be defined variously. In any given computation of CNS the power vectors for the social interaction event and the expectation baseline should be defined consistently with one another, and the weighting vectors should be identical. A power vector may include arousal measures only, valence values only, a combination of arousal measures and valence measures, or a combination of any of the foregoing with other measures, for example a confidence measure. A processor may compute multiple different power vectors for the same user at the same time, based on different combinations of sensor data, expectation baselines, and weighting vectors. In one embodiment, CNS is calculated using power vectors [symbol] defined by a combination of ‘j’ arousal measures ‘aⱼ’ and ‘k valence measures ‘vₖ’, each of which is adjusted by a calibration offset ‘C’ from a known stimulus, wherein j and k are any non-negative integer, as follows:

[symbol]

=(a₁C₁, . . . ,aⱼCⱼ, . . . ,vₖCⱼ₊ₖ) Eq. 5

wherein

Cⱼ=Sⱼ−SⱼOⱼ=Sⱼ(1−Oⱼ) Eq. 6

The index ‘j’ in Equation 6 signifies an index from 1 to j+k, Sⱼ signifies a scaling factor and Oⱼ signifies the offset between the minimum of the sensor data range and its true minimum. A weighting vector [symbol] corresponding to the power vector of Equation 5 may be expressed as:

[symbol]

=(w₁, . . . ,wⱼ,wⱼ₊₁, . . . wₖ) Eq. 7

wherein each weight value scales its corresponding factor in proportion to the factor's relative estimated reliability.

With calibrated dot products Pᵥ_{ₙ}, Pₓ_{ₘ} given by Equations 3 and 4 and time factors as given by Equations 1 and 2, a processor may compute a Composite Neuro-physiological State (CNS) for a single user as follows:

$${CNS}_{user} ( dBm ) = 10. {log}_{10} ( \frac{{\sum }_{n}^{1} {P}_{v} Δ {t}_{ν}}{{\sum }_{m}^{1} {P}_{x} Δ {t}_{x}} . \frac{{t}_{x}}{{t}_{ν}} ) \qquad Eq . 8$$

The ratio

$$\frac{{t}_{x}}{{t}_{v}}$$

normanzes inequality in me aisparate time series sums and renders the ratio unitless. A user CNS value greater than 1 indicates that a user/player actor/viewer is experiencing a neuro-physiological response greater than baseline for the type of social interaction at issue. A user CNS value less than 1 indicates a neuro-physiological response less than baseline for the type of social interaction. A processor may compute multiple different CNS values for the same user at the same time, based on different power vectors.

Equation 5 describes a calibrated power vector made up of arousal and valence measures derived from biometric sensor data. In an alternative, the processor may define a partially uncalibrated power vector in which the sensor data signal is scaled as part of lower-level digital signal processing before conversion to a digital value but not offset for a user as follows:

[symbol]

=(aᵢ, . . . ,aⱼ,v, . . . ,vₖ) Eq. 9

If using a partially uncalibrated power vector, an aggregate calibration offset may be computed for each factor and subtracted from the dot products Pᵥ_{ₙ}, Pₓ_{ₘ} given by Equations 3 and 4 before calculating Composite Neuro-physiological State (CNS). For example, an aggregate calibration offset for Pᵥ_{ₙ} may be given by:

Cᵥ=i( [symbol] · [symbol] )=iΣᵢ¹Cᵥ_{ᵢ}Wᵢ=Cᵥ_{₁}W₁+Cᵥ_{₂}W₂+ . . . +Cᵥ_{ᵢ}Wᵢ Eq. 10

In such case, a calibrated value of the power vector Pᵥ_{ₙ} can be computed by:

pᵥ_{ₙ}−Cᵥ_{ₙ} Eq. 11

The calibrated power vector Pₓ_{ₘ} can be similarly computed.

Referring again to the method 800 in which the foregoing expressions can be used (FIG. 8), a calibration process 802 for the sensor data is first performed to calibrate user reactions to known stimuli, for example a known resting stimulus 804, a known arousing stimulus 806, a known positive valence stimulus 808, and a known negative valence stimulus 810. The known stimuli 806-810 can be tested using a focus group that is culturally and demographically like the target group of users and maintained in a database for use in calibration. For example, the International Affective Picture System (ZAPS) is a database of pictures for studying emotion and attention in psychological research. For consistency with the content platform, images of these found in the IAPS or similar knowledge bases may be produced in a format consistent with the targeted platform for use in calibration. For example, pictures of an emotionally-triggering subject can be produced as video clips. Calibration ensures that sensors are operating as expected and providing data consistently between users. Inconsistent results may indicate malfunctioning or misconfigured sensors that can be corrected or disregarded. The processor may determine one or more calibration coefficients 816 for adjusting signal values for consistency across devices and/or users.

Calibration can have both scaling and offset characteristics. To be useful as an indicator of arousal, valence, or other psychological state, sensor data may need calibrating with both scaling and offset factors. For example, GSR may in theory vary between zero and 1, but in practice depend on fixed and variable conditions of human skin that vary across individuals and with time. In any given session, a subject's GSR may range between some GSRₘᵢₙ>0 and some GSRₘₐₓ<1. Both the magnitude of the range and its scale may be measured by exposing the subject to known stimuli and estimating the magnitude and scale of the calibration factor by comparing the results from the session with known stimuli to the expected range for a sensor of the same type. In many cases, the reliability of calibration may be doubtful or calibration data may be unavailable, making it necessary to estimate calibration factors from live data. In some embodiments, sensor data might be pre-calibrated using an adaptive machine learning algorithm that adjusts calibration factors for each data stream as more data is received and spares higher-level processing from the task of adjusting for calibration.

Once sensors are calibrated, the system normalizes the sensor data response data for genre differences at 812, for example using Equation 8. Different types of social interactions produce different valence and arousal scores. For example, first-person shooter games have a different pace, focus, and intensity from online Poker or social chat. Thus, engagement power cannot be compared across different application types unless the engagement profile of the application type is considered. Genre normalization scores the application relative to applications of the same type, enabling comparison on an equivalent basis across genres. Normalization 812 may be performed on a user or users before beginning play. For example, users may play a trial, simulated game before the real game, and a processor may use data from the simulated game for normalization. In an alternative, a processor may use archived data for the same users or same user cohort to calculate expectation power. Expectation power is calculated using the same algorithms as used or that will be used for measurements of event power and can be adjusted using the same calibration coefficients 816. The processor stores the expectation power 818 for later use.

At 820, a processor receives sensor data during play of the subject content and calculates event power for each measure of concern, such as arousal and one or more valence qualities. At 828, the processor sums or otherwise aggregates the event power for the content after play is concluded, or on a running basis during play. At 830, the processor calculates a representation of the user's neuro-physiological state, for example, Composite Neuro-physiological State (CNS) as previously described. The processor first applies applicable calibration coefficients and then calculates the CNS by dividing the aggregated event power by the expectation power as described above.

Optionally, the calculation function 820 may include comparing, at 824, an event power for each detected event, or for a lesser subset of detected events, to a reference for a social/game experience. A reference may be, for example, a baseline defined by a game designer or by the user's prior data. For example, in Poker or similar wagering games, bluffing is a significant part of game play. A game designer may compare a current event power (e.g., measured when a user is placing a bet) with a baseline reference (e.g., measured between hands or prior to the game). At 826, the processor may save, increment or otherwise accumulate an error vector value describing the error for one or more variables. The error vector may include a difference between the references and a measured response for each measured value (e.g., arousal and valence values) for a specified event or period of a social interaction. The error vector and matrix of vectors may be useful for content evaluation or content control.

Error measurements may include or augment other metrics for content evaluation. Composite Neuro-physiological State and error measurements may be compared to purchases, subscriptions, or other conversions related to presented content. The system may also measure consistency in audience response, using standard deviation or other statistical measures. The system may measure Composite Neuro-physiological State, valence and arousal for individual, cohorts, and aggregate audiences. Error vectors and CNS may be used for a variety of real-time and offline tasks.

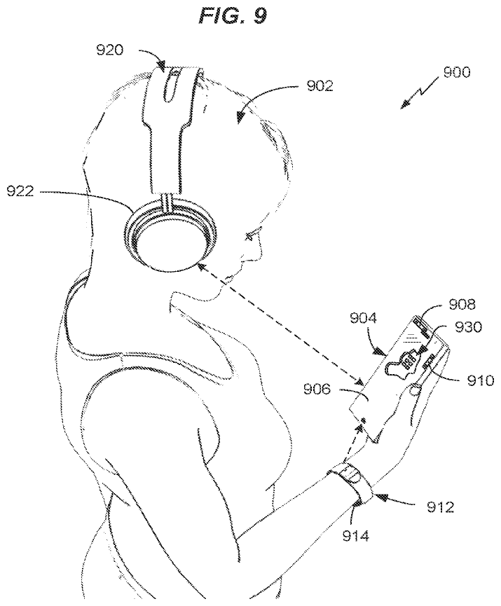

FIG. 9 shows a mobile system 900 for a user 902 including a mobile device 904 with sensors and accessories 912, 920 for collecting biometric data used in the methods and apparatus described herein and a display screen 906. The mobile system 900 may be useful real-time or non-real-time control of applications such as traditional content-wide focus group testing. The mobile device 904 may use built in sensors commonly included on consumer devices (phones, tables etc.) for example a front facing stereoscopic camera 908 (portrait) or 910 (landscape). Often included by manufacturers for face detection identity verification, cameras 908, 910 may also be used for eye tracking for tracking attention, FAU for tracking CNS-valence, pupil dilation measurement tracking CNS-arousal and heartrate as available through watch accessory 912 including a pulse detection sensor 914, or by the mobile device 904 itself.

Accessories like a headphone 920, hats or VR headsets may be equipped with EEG sensors 922. A processor of the mobile device may detect arousal by pupil dilation via the 3D cameras 908, 910 which also provide eye tracking data. A calibration scheme may be used to discriminate pupil dilation by aperture (light changes) from changes due to emotional arousal. Both front and back cameras of the mobile device 904 may be used for ambient light detection, and for calibration of pupil dilation detection factoring out dilation caused by lighting changes. For example, a measure of pupil dilation distance (mm) versus dynamic range of light expected during the performance for anticipated ambient light conditions may be made during a calibration sequence. From this, a processor may calibrate out effects from lighting vs. effect from emotion or cognitive workload based on the design of the narrative by measuring the extra dilation displacement from narrative elements and the results from the calibration signal tests.

Instead of, or in addition to a stereoscopic camera 908 or 910, a mobile device 904 may include a radar sensor 930, for example a multi-element microchip array radar (MEMAR), to create and track facial action units and pupil dilation. The radar sensor 930 can be embedded underneath and can see through the display screen 906 on a mobile device 904 with or without visible light on the subject. The display screen 906 is invisible to the RF spectrum radiated by the imaging radar arrays, which can thereby perform radar imaging through the screen in any amount of light or darkness. In an aspect, the MEMAR sensor 930 may include two arrays with 6 elements each. Two small RF radar chip antennas with six elements each create an imaging radar. An advantage of the MEMAR sensor 930 over optical sensors in the cameras 908, 910 is that illumination of the face is not needed, and thus sensing of facial action units, pupil dilation and eye tracking is not impeded by darkness. While only one 6-chip MEMAR array 930 is shown, a mobile device may be equipped with two or more similar arrays for more robust sensing capabilities.

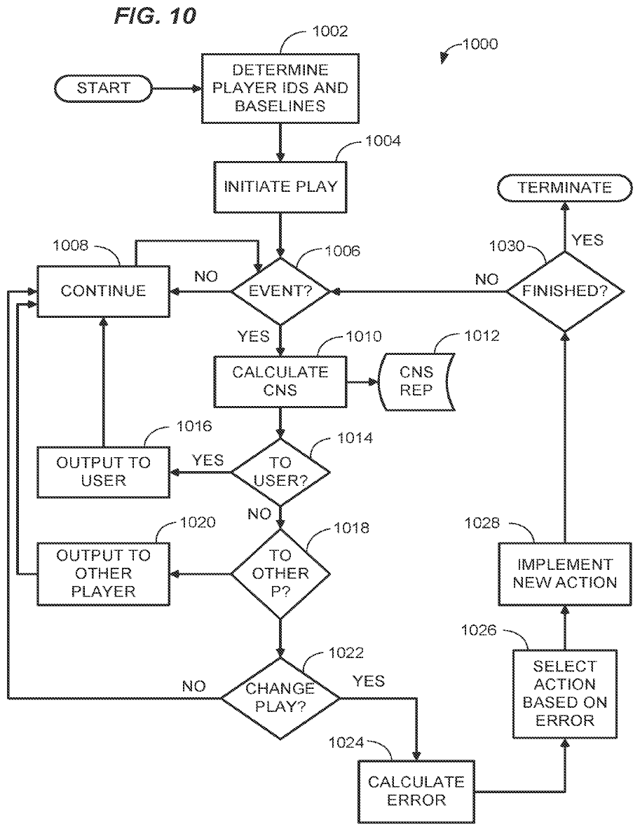

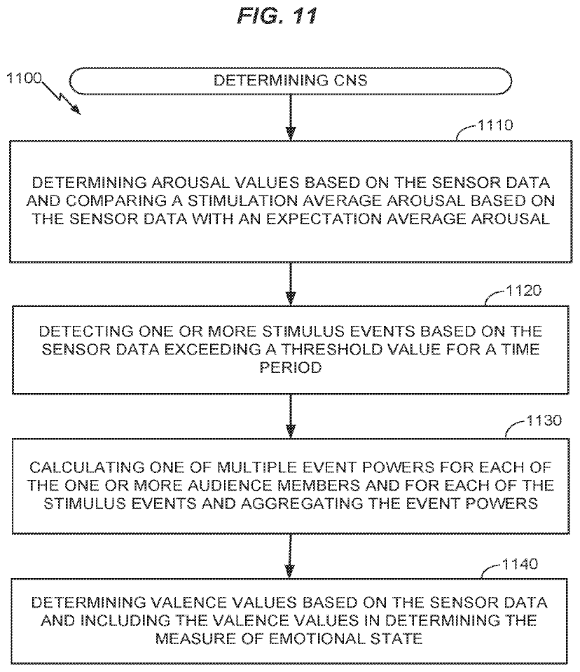

FIG. 10 illustrates aspects of a method 1000 for controlling a social interaction application using biometric sensor data. The method may apply to various different games and ways of social interaction, in which any one or more of the following occurs: (1) the user receives an indication of one or more CNS measurements for himself or herself; (2) other players, spectators or monitors receive an indication of the user's CNS measurements, or (3) the application changes operating parameters, game play, or takes some other action in response to comparing the user's CNS measurements to a baseline. A processor executing code for performing the method 1000 may trigger measurement of CNS based on occurrence of an event, e.g., an event trigger (e.g., an event significant to the progress or outcome of the social interaction application is triggered, such as a wager is placed or raised, a non-player character or other player challenges the user's avatar, a contest event begins, a contest event ends, a social interaction occurs, etc.), a passage of time (e.g., every fifteen seconds during play), or a biometric sensor input (e.g., an occurrence of a measure or indication of one or more biometric inputs exceeding a fixed predetermined data threshold or a variable number such as a rolling average). In an aspect, the measurement of CNS may be triggered based on a combination of the foregoing. The method 1000 is generic to at least the types of social interaction applications described below, but is not limited to the applications described below:

The method 1000 may be used for competitive bluffing games with or without monetary wagers, for example Poker, Werewolf™, Balderdash™ and similar games. In these games, players compete to fool other players. The method may be used in a training mode wherein only the user sees his or her own CNS indicators, in a competitive mode wherein every player sees the other players' CNS indicators, in a perquisite mode wherein players may win or be randomly awarded access to another player's CNS indicators, in an interactive mode wherein the processor modifies game play based on one or more players' CNS indicators, a spectator mode in which CNS values are provided to spectators, or any combination of the foregoing.

The method 1000 may be used for any game or other social interaction to improve the user experience in response to CNS indicators. For example, if CNS indicators show frustration, the processor may ease game play; if the indicators show boredom, the processor may introduce new elements, change technical parameters of the game affecting appearance and pacing, or provide a difference challenge. In an aspect, a processor may apply a machine learning algorithm to optimize any desired parameter (e.g., user engagement) based on correlating CNS data to game play.

The method 1000 may be used in social games involving sharing preferences for any subject matter, including, for example, picking a preferred friend or date; choosing a favorite item of clothing or merchandise, meme, video clip, photograph or art piece or other stimulus, with or without revealing a user's CNS data to other players. Such social games may be played with or without a competitive element such as electing a most favored person or thing.

The method 1000 may be used in social games for enhancing interpersonal communication, by allowing participants to better understand the emotional impact of their social interactions, and to adjust their behavior accordingly.

The method 1000 may be used in social games in which, like bluffing, the object includes concealing the player's emotional state, or in games in which the object includes revealing the player's emotional state. In either case, the CNS data may provide a quantitative or qualitative basis for comparing the performances of different players.

The method may be used in athletic contests. A processor may provide the CNS to a device belonging to each competitor or competitor's team for managing play. In an alternative, or in addition, a processor may provide the CNS to a device belonging to one or more referees or spectators to improve safety or enjoyment of the contest. In an alternative, or in addition, a processor may provide the CNS to a device belonging to an opponent or the opponent's team, to enable new styles of play.

The method 1000 may include, at 1002 a processor determining, obtaining, or assigning one or more player's identifications and corresponding baseline neuro-physiological responses to stimuli that simulate one or more social interactions that may occur during a social interaction application. For example, in an aspect, the baselines may include baseline arousal and valence values. In an aspect, the baseline neuro-physiological responses may be obtained from a database of biometric data (e.g., 610: FIG. 6), and it may be specific to a given player (to the extent the database already contains baseline data previously obtained from the player), or alternatively, a set of generic baseline data may be assigned to the specific player based on a set of baseline data attributable to the cultural or demographic category to which the player belongs, or the baseline data may be randomly assigned, or by other suitable means. In an aspect, the baselines may be determined during emotion calibration as previously discussed herein. Such baseline determination, however, is not always necessary for each and every player, nor is it necessary for each play session of a game or another social interaction application contemplated by the present disclosure.

At 1004, the processor initiates the play of the social interaction application in which the one or more players (including human and/or computer players) participate. At 1006, the processor determines whether an event as previously described herein has occurred, such that a measurement of CNS would be triggered. To do so, for example, the processor may monitor the behavior or neuro-physiological state of the one or more players, using sensors and client devices as described herein. For example, the behavior of players may be monitored using sensors described below with respect to the example of an implementation in a game room of a casino in the paragraph immediately below. If no event is detected, at 1008, the processor continues to wait until one is detected. If an event is detected, at 1010, the processor proceeds to calculate the measurement of the CNS value for the one or more players.

For example, in the method 1000 involving a game of Poker among a first player and two or more other players (including a dealer), suppose the first player is a player “under the gun” (meaning required to match another player's bet or leave the game) and immediately following the player that has posted a big blind in the amount of $75. The hand begins, and the dealer deals two down cards to each player. The first player under the gun calls and raises the bet by placing chips in the amount greater than the big blind, e.g., $5000, as detected by the system and sensors described herein. In such case, the processor at 1006 determines that an event (e.g., a wager is raised) has occurred. At 1010, the processor calculates the measurement of CNS value for the first player upon the event, and at 1012, the processor stores a set of data that represents the measured CNS in a memory.

In an aspect, at 1014, the processor determines whether to output the calculated CNS to the first player. For example, suppose the hand in the just-described game of Poker was previously designated as a training session player by the first player training against a computer algorithm. In such case, the processor determines that the CNS calculated at 1010 should be outputted to the first player at 1014.

At 1016, the first player may perceive or sense the output of the calculated CNS in any one or more of suitable qualitative or quantitative forms, including, for example, digital representations (e.g., numerical values of arousal or valence or other biometric data such as temperature, perspiration, facial expressions, postures, gestures, etc.), percentages, colors, sounds (e.g., audio feedback, music, tactile feedbacks, etc. For example, suppose the first player was bluffing when he raised the bet to $5000, and the first player has exhibited neuro-physiological signs detectable by biometric sensors of the present disclosure consistent with an event of bluffing. In such case, in an implementation of the training mode, the processor may provide to the first player an audio feedback, “bluffing,” a recognizable tactile feedback suggesting to the player that the bluff has been detected, an alert message on a display showing the player with a text, “bluffing,” and the like.

When the processor determines that the calculated CNS should not be outputted to the first player, the processor at 1018 determines whether the calculated CNS should be outputted to other players. For example, continuing the example of the Poker game in training mode, wherein at 1014 the processor has determined that the first player is bluffing, but in an alternative training mode where the detection of bluffing is not revealed or outputted to the first player, the processor may instead output the calculated CNS to other players, as part of the training mode programming. At 1020, the calculated CNS may be outputted to the other players, similar in manner as the case for the first player in 1016.

At 1022, the processor may change the play of the social interaction application. For example, continuing the example of the game of Poker in training mode, the processor may determine the course of action of one or more computer algorithm players participating in the Poker game, after the bluffing by the first player is detected as described above. For example, suppose the computer algorithm player, prior to the first player raising the bet, was prepared to call the bet by matching the big blind ($75). Instead, the processor changes the play by calling the bet of the first player and raising it to $5100.

At 1024, the processor may calculate error vector of the measurement of CNS. For example, continuing the example of the Poker game, assume that at the end of the entire hand, the first player, whom the processor previously determined as “bluffing,” turns out winning the round. Then, at 1024, the processor calculates the error vector for the “bluffing” determination. At 1026, the processor selects an action based on the calculated error. For example, continuing the example of the Poker game, the processor at 1026 may update the “bluffing” parametric values, and for the same set of biometric data previously flagged as “bluffing,” the processor would no longer deem the set as “bluffing.” At 1028, the processor may implement a new action. For example, continuing the Poker game example where the parameters for detecting “bluffing” has been updated, in a future round of Poker game in which the first player participates, the processor would not deem the same set of biometric data previously flagged as “bluffing” as such, and instead, the computer algorithm player may, for example, decide to fold in such in case the same set of biometric data is detected from the first player.

The operation 1024 may be performed for other reasons, also. For example, in a social introduction game, the processor may determine based on a high error value that one or more participants in a social introduction session is uneasy. Then, in 1026, the processor may select an operation to reduce the detected discomfort. For example, the processor may execute an intervention script to detect and reduce the source of uneasiness, up to and including at 1028 removing a participant from the session and placing removed participants in a new session with different people. For further example, if the processor determines that a player of an action game is frustrated or bored, it may reduce or increase the level of challenge presented by the game to increase the player's interest and time of play.

At 1030, the processor monitors whether the social interaction application is finished. For example, continuing the example of the Poker game, when the first player playing against other computer algorithm players leaves the table or otherwise ends participating in the game, the game is terminated.

Specific embodiments of the method 1000 may include using biometric feedback to improve the accuracy of estimates of player intent in casino games involving obfuscation of the strength of a player's standing, including anticipation of bets, raises, and bluffs. Systems and sensors as described herein may be used to record biometrics and/or player behaviors, gestures, postures, facial expressions, and other biometric indicators, through video and audio capture, thermal imaging, breath monitoring and other biometrics while a player is engaged in a casino game. A processor may record the CNS score in reference to calibration with emotional feedback when subjects are calm compared to when they are bluffing, or otherwise engaging in acts of deceit. For example, an implementation in a game room of a casino or the like may include: 1) Deploying front facing stereo camera (eyetracking, pupil dilation, FAU), microphone (Audio speech analysis, NLP word analysis), phased array sensor (eyetracking, pupil dilation, FAU), IR sensor (fNIR), laser breath monitor in a casino setting at Poker and Poker-derivative games involving bets and bluffing, and providing real-time analysis and feedback to casino managers and dealers; 2) Improving upon Poker-playing computer algorithms to provide missing information about human opponents' biometric status, and 3) Using machine learning to allow a Poker playing computer to detect human intent, anticipation of bets, raises, and bluffs. Other applications may include, for example, deploying Poker playing computers against human champion Poker players in a tournament setting, i.e. to test ultimate human vs. computer Poker skills. In some implementations, a provider may package a hardware kit including stereo cameras, microphones, phased array, IR and laser sensors for use by Poker playing professionals to train against a computer algorithm that uses biometrics to detect human intent for the purposes of improving their game.

Other applications may use biometric feedback in a strategy game involving obfuscation of the strength of a player's standing, for example, regarding military unit or equipment strength, anticipation of attacks, retreats, ruses, ambushes and bluffs. Availability of biometric feedback for all the players in the strategy game may be provided to human or computer opponents to enhance the determined accuracy of an opponent or opponents' state and intent, to increase the challenge of the game or to offer new forms of play based entirely around bluffing.

Referring to FIG. 11 showing certain additional operations or aspects 1100 for signaling users or others during participation in a social interaction application, the method 1000 may further include, at 1110, determining the measure of composite neuro-physiological state at least in part by determining arousal values based on the sensor data and comparing a stimulation average arousal based on the sensor data with an expectation average arousal. For example, the CNS includes a measure of arousal and valence. Non-limiting examples of suitable sensors for detecting arousal are listed above in connection with FIGS. 4 and 10.

In a related aspect, the method 1000 may include, at 1120, determining the measure of composite neuro-physiological state at least in part by detecting one or more stimulus events based on the sensor data exceeding a threshold value for a time period. In a related aspect, the method 1000 may include, at 1130, calculating one of multiple event powers for each of the one or more audience members and for each of the stimulus events and aggregating the event powers. In an aspect, the method 1000 may include assigning, by the at least one processor, weights to each of the event powers based on one or more source identities for the sensor data At 1140, the method 1000 may further include determining the measure of composite neuro-physiological state at least in part by determining valence values based on the sensor data and including the valence values in determining the measure of composite neuro-physiological state. A list of non-limiting, example suitable sensors is provided above in connection with FIGS. 4 and 10.

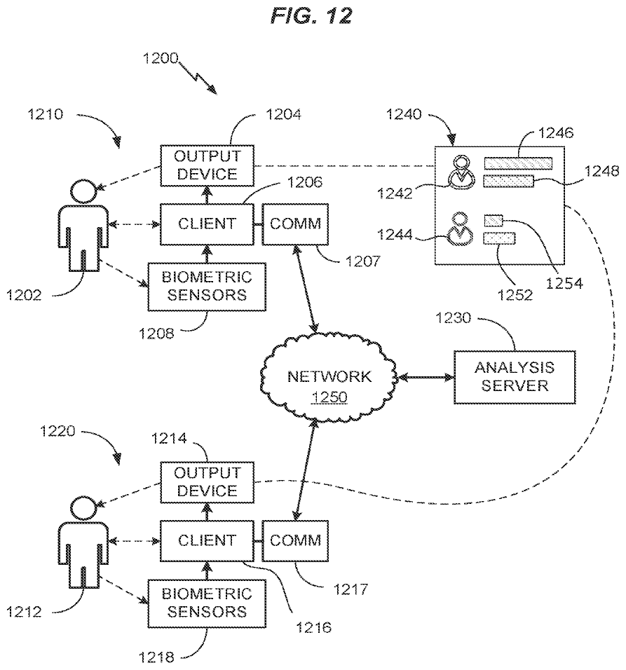

The method and apparatus described herein for controlling a social interaction application production may be adapted for improving person-to-person communication in virtual or real environments. FIG. 12 shows a system 1200 including a first node 1210 with a first person 1202 in communication with a second node 1220 with a second person 1212 via an electronic communication network 1250. The system 1200 may use a CNS model for communication where CNS values are presented and measured alongside a conversation. For example, the two people 1202, 1212 can converse while one or more of the participating clients 1206, 1216 present data on emotional affect alongside the photo or video 1242, 1244 of each participant. Neuro-physiological responses of the participants 1202, 1212 are sensed using corresponding biometric sensors 1208, 1218 and described elsewhere herein. Each client 1206, 1216 may convert sensor signals from the biometric sensors 1208, 1218 into biometric data and send the biometric data to an analysis server 1230 via respective communication components 1207, 1217 and a communication network 1250. The server 1230 may generate in real time or near real time one or more measures of valence, arousal, dominance, CNS or any other suitable measure of neuro-physiological response, and provide the one or more measures via the network 1250 to the clients 1206, 1216.

Each client 1206, 1216 may output the measures via output devices 1204, 1214, for example a display screen, as a graphical display 1240 or other useful format (e.g., audible output). The display 1240 or other output may report neuro-physiological state measures for conversation sequence statements or groups of statements. For example, a display 1240 may include an indication of arousal 1246, 1254 or valence 1248, 1252. The system 1200 may provide an alert any time there's a rapid increase in arousal and also report the valence associated with the increase. The alert can then be appraised by the human for meaning. The system 1200 may be especially useful for human to human communication between players actors within a virtual immersive experience and may find application in other contexts also.

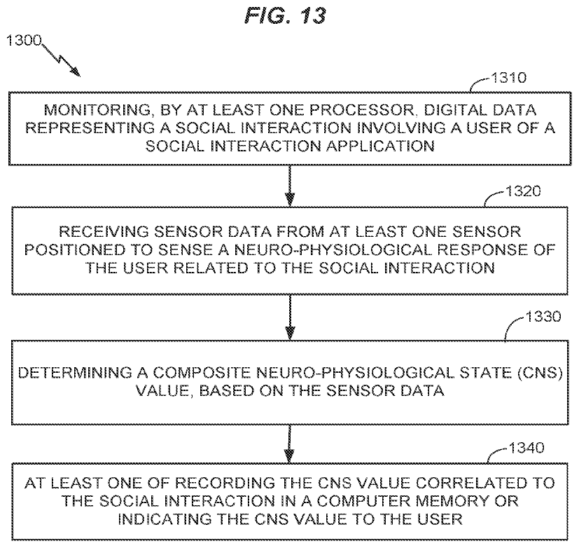

In view the foregoing, and by way of additional example, FIGS. 13-16 show aspects of a method 1300 or methods for controlling a social interaction application based on a representation of a neuro-physiological state of a user. In some aspect, the social interaction application may be one or more of a card game, a bluffing game, a dating application, a social networking application, an action video game, an adventure video game, a role-playing video game, a simulation video game, a strategy video game, a sports video game and a party video game. The method 1300 may be performed by an immersive mixed reality output device or a non-immersive flat screen device, projector, or other output device including a programmable computer, by one or more computers in communication with the output device, or by a combination of an output device and one or more computers in communication with the output device.

Referring to FIG. 13, a computer-implemented method for controlling a social interaction application based on a representation of a neuro-physiological state of a user may include, at 1310, monitoring, by at least one processor, digital data from a social interaction involving a user of the application. The digital data may be encoded for an output device, for example, a portable or non-portable flat screen device, a digital projector, or wearable gear for alternative reality or augmented reality, in each case coupled to an audio output capability and optionally to other output capabilities (e.g., motion, tactile, or olfactory). Playing the digital data may include, for example, keeping the digital data in a cache or other memory of the output device and processing the data for output by at least one processor of the output device. The digital data may represent a state of the social interaction or social interaction application, for example, a game state, a record of chat or other social interaction, or other data for correlating to a neuro-physiological response of one or more participants in the social interaction.

The method 1300 may include, at 1320, receiving sensor data from at least one sensor positioned to sense a neuro-physiological response of the user related to the social interaction. The sensor data may include any one or more of the data described herein for arousal, valence, or other measures.

The method 1300 may include at 1330 determining a Composite Neuro-physiological State (CNS) value for the social interaction, based on the sensor data, using an algorithm as described herein above. In an alternative, the method may determine a different measure for neuro-physiological response. The method may include at 1340 recording the CNS value or other neuro-physiological measure correlated to the social interaction in a computer memory. In an alternative, the method may include indicating the CNS value or other neuro-physiological measure to the user and/or recipient. In an alternative, the method may include controlling progress of the social interaction application based at least in part on the CNS value.

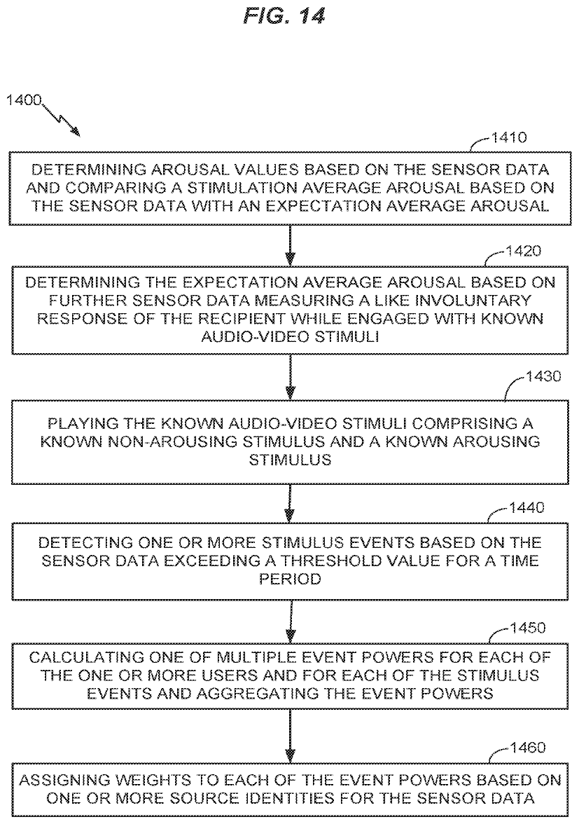

FIGS. 14-16 list additional operations 1400, 1500, 1600 that may be performed as part of the method 1300. The elements of the operations 1400, 1500, 1600 may be performed in any operative order, and any one or any number of them may be omitted from the method 1300.

Referring to FIG. 14, the method 1300 may include any one or more of the additional operations 1400 for determining a CNS value. The method 1300 may include, at 1410 determining the CNS value at least in part by determining arousal values based on the sensor data and comparing a stimulation average arousal based on the sensor data with an expectation average arousal. The sensor data for arousal may include one or more of electroencephalographic (EEG) data, galvanic skin response (GSR) data, facial electromyography (fEMG) data, electrocardiogram (EKG) data, video facial action unit (FAU) data, brain machine interface (BMI) data, video pulse detection (VPD) data, pupil dilation data, functional magnetic resonance imaging (fMRI) data, and functional near-infrared data (fNIR). The method 1300 may include, at 1420, determining the expectation average arousal based on further sensor data measuring a like involuntary response of the recipient while engaged with known audio-video stimuli.

In another aspect, the method 1300 may include, at 1430 playing the known audio-video stimuli comprising a known non-arousing stimulus and a known arousing stimulus. The method 1300 may include, at 1440 determining the CNS value at least in part by detecting one or more stimulus events based on the sensor data exceeding a threshold value for a time period. The method 1300 may include, at 1450 calculating one of multiple event powers for each of the one or more users and for each of the stimulus events and aggregating the event powers. The method 1300 may include, at 1460 assigning weights to each of the event powers based on one or more source identities for the sensor data.

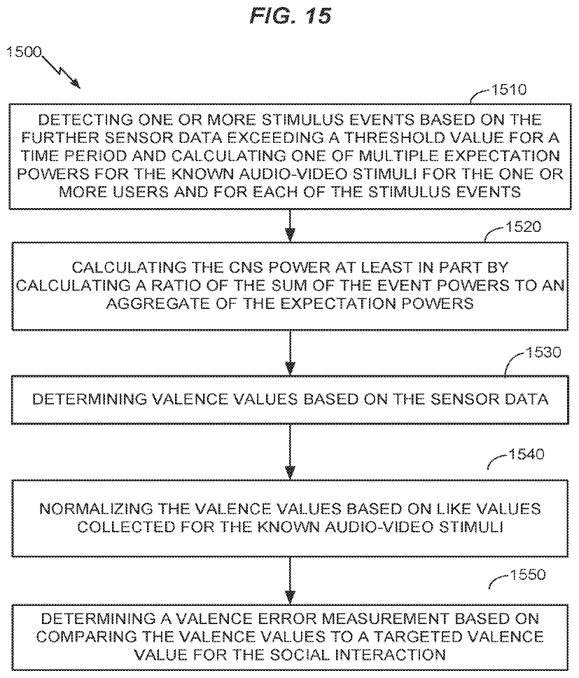

Referring to FIG. 15, the method 1300 may include any one or more of the additional operations 1500 for determining a CNS value. The method 1300 may include, at 1510 determining the expectation average arousal at least in part by detecting one or more stimulus events based on the further sensor data exceeding a threshold value for a time period and calculating one of multiple expectation powers for the known audio-video stimuli for the one or more users and for each of the stimulus events. The method 1300 may include, at 1520 calculating the CNS power at least in part by calculating a ratio of the sum of the event powers to an aggregate of the expectation powers.

In a related aspect, the method 1300 may include, at 1530 determining valence values based on the sensor data. The sensor data for valence may include one or more of electroencephalographic (EEG) data, facial electromyography (fEMG) data, video facial action unit (FAU) data, brain machine interface (BMI) data, functional magnetic resonance imaging (fMRI) data, functional near-infrared data (fNIR) and positron emission tomography (PET). The method 1300 may include, at 1540 normalizing the valence values based on like values collected for the known audio-video stimuli. The method 1300 may include, at 1550 determining a valence error measurement based on comparing the valence values to a targeted valence for the social interaction.

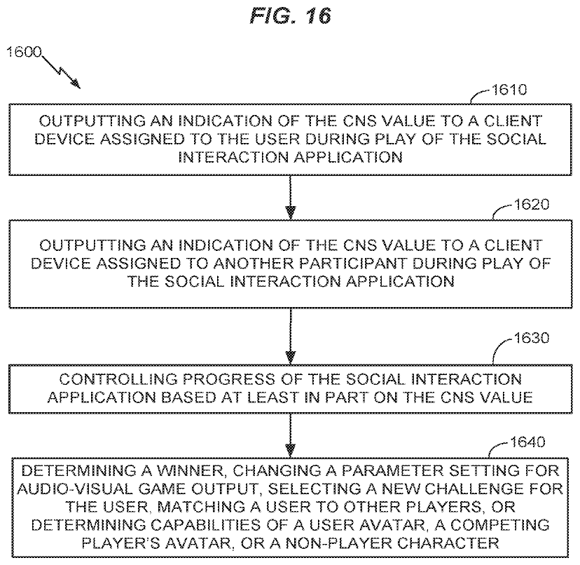

Referring to FIG. 16, the method 1300 may include any one or more of the additional operations 1600 for determining a CNS value. The method 1300 may include, at 1610, outputting an indication of the CNS value to a client device assigned to the user during play of the social interaction application. The method may include, at 1620, outputting an indication of the CNS value to a client device assigned to another participant during play of the social interaction application. The method may include, at 1630, controlling progress of the social interaction application based at least in part on the CNS value. For example, at 1640, controlling progress of the social interaction application may include at least one of: determining a winner, changing a parameter setting for audio-visual game output, selecting a new challenge for the user, matching a user to other players, or determining capabilities of a user avatar, a competing player's avatar, or a non-player character.

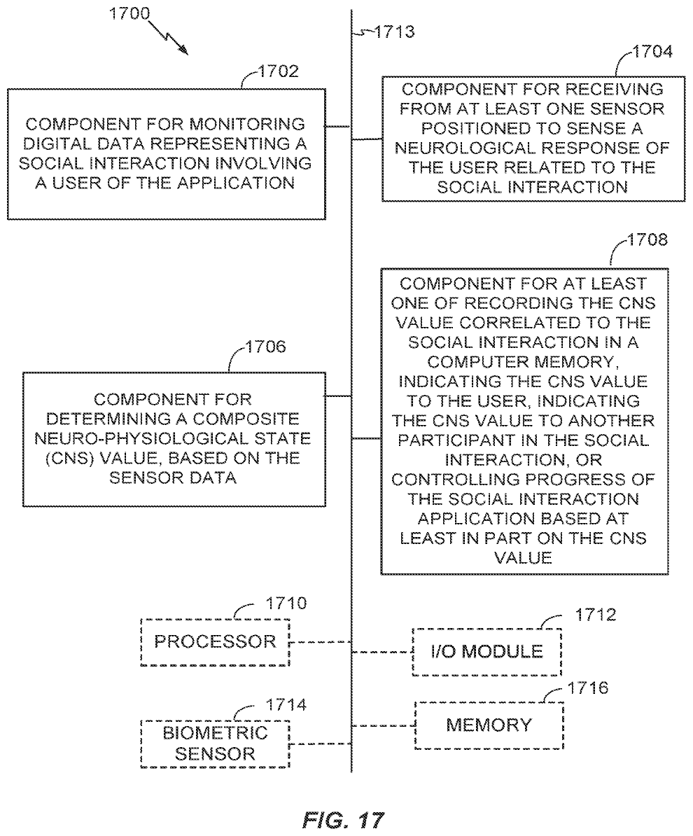

FIG. 17 is a conceptual block diagram illustrating components of an apparatus or system 1700 for controlling a social interaction application based on a representation of a neuro-physiological state of a user. The apparatus or system 1700 may include additional or more detailed components for performing functions or process operations as described herein. For example, the processor 1710 and memory 1716 may contain an instantiation of a process for calculating CNS in real time as described herein above. As depicted, the apparatus or system 1700 may include functional blocks that can represent functions implemented by a processor, software, or combination thereof (e.g., firmware).

As illustrated in FIG. 17, the apparatus or system 1700 may comprise an electrical component 1702 for monitoring, by at least one processor, digital data from a social interaction involving a user of the application. The component 1702 may be, or may include, a means for said monitoring. Said means may include the processor 1710 coupled to the memory 1716, and to an output of at least one biometric sensor 1714, the processor executing an algorithm based on program instructions stored in the memory. Such algorithm may include, for example, detecting a context of a social interaction, including that the social interaction is directed to eliciting a targeted neuro-physiological response, and creating an association between the social interaction and the targeted response.

The apparatus 1700 may further include an electrical component 1704 for receiving sensor data from at least one sensor positioned to sense a neuro-physiological response of the user related to the social interaction. The component 1704 may be, or may include, a means for said receiving. Said means may include the processor 1710 coupled to the memory 1716, the processor executing an algorithm based on program instructions stored in the memory. Such algorithm may include a sequence of more detailed operations, for example, configuring a data port to receive sensor data from a known sensor, configuring a connection to the sensor, receiving digital data at the port, and interpreting the digital data as sensor data.

The apparatus 1700 may further include an electrical component 1706 for determining a Composite Neuro-physiological State (CNS) value for the social interaction, based on the sensor data. The component 1706 may be, or may include, a means for said determining. Said means may include the processor 1710 coupled to the memory 1716, the processor executing an algorithm based on program instructions stored in the memory. Such algorithm may include a sequence of more detailed operations, for example, as described in connection with FIG. 8.

The apparatus 1700 may further include an electrical component 1708 for at least one of recording the CNS value correlated to the social interaction in a computer memory or indicating the CNS value to the user, indicating the CNS value to another participant in the social interaction, or controlling progress of the social interaction application based at least in part on the CNS value. The component 1708 may be, or may include, a means for said recording or indicating. Said means may include the processor 1710 coupled to the memory 1716, the processor executing an algorithm based on program instructions stored in the memory. Such algorithm may include a sequence of more detailed operations, for example, encoding the CNS value and storing the encoded value in a computer memory, or sending the encoded value to an output device for presentation to the user.

The apparatus 1700 may optionally include a processor module having at least one processor 1710. The processor 1710 may be in operative communication with the modules 1702-1708 via a bus 1713 or similar communication coupling. In the alternative, one or more of the modules may be instantiated as functional modules in a memory of the processor. The processor 1710 may initiate and schedule the processes or functions performed by electrical components 1702-1708.

In related aspects, the apparatus 1700 may include a network interface module 1712 or equivalent I/O port operable for communicating with system components over a computer network. A network interface module may be, or may include, for example, an Ethernet port or serial port (e.g., a Universal Serial Bus (USB) port), a Wi-Fi interface, or a cellular telephone interface. In further related aspects, the apparatus 1700 may optionally include a module for storing information, such as, for example, a memory 1716. The computer readable medium or the memory 1716 may be operatively coupled to the other components of the apparatus 1700 via the bus 1713 or the like. The memory 1716 may be adapted to store computer readable instructions and data for effecting the processes and behavior of the modules 1702-1708, and subcomponents thereof, or the processor 1710, the method 1300 and one or more of the additional operations 1400-1600 disclosed herein, or any method for performance by a media player described herein. The memory 1716 may retain instructions for executing functions associated with the modules 1702-1708. While shown as being external to the memory 1716, it is to be understood that the modules 1702-1708 can exist within the memory 1716 or an on-chip memory of the processor 1710.

The apparatus 1700 may include, or may be connected to, one or more biometric sensors 1714, which may be of any suitable types. Various examples of suitable biometric sensors are described herein above. In alternative embodiments, the processor 1710 may include networked microprocessors from devices operating over a computer network. In addition, the apparatus 1700 may connect to an output device as described herein, via the I/O module 1712 or other output port.

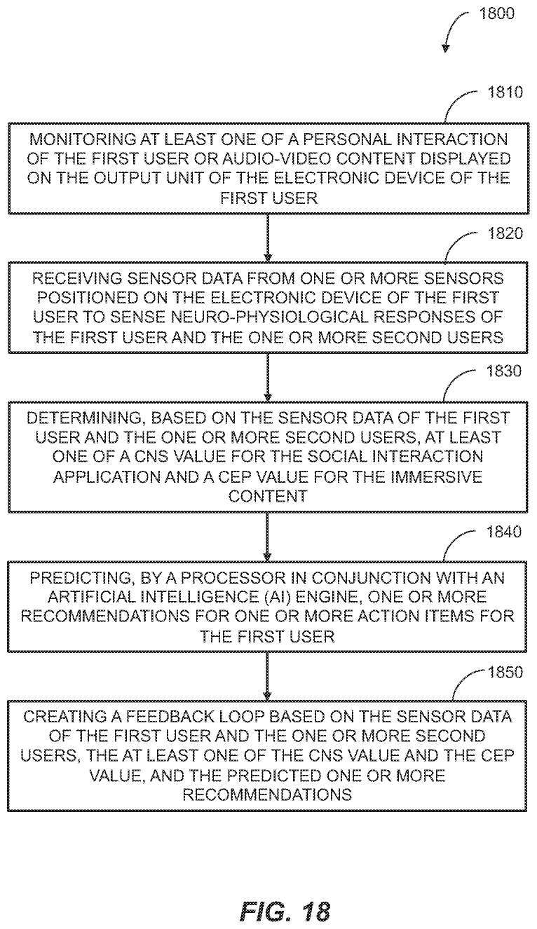

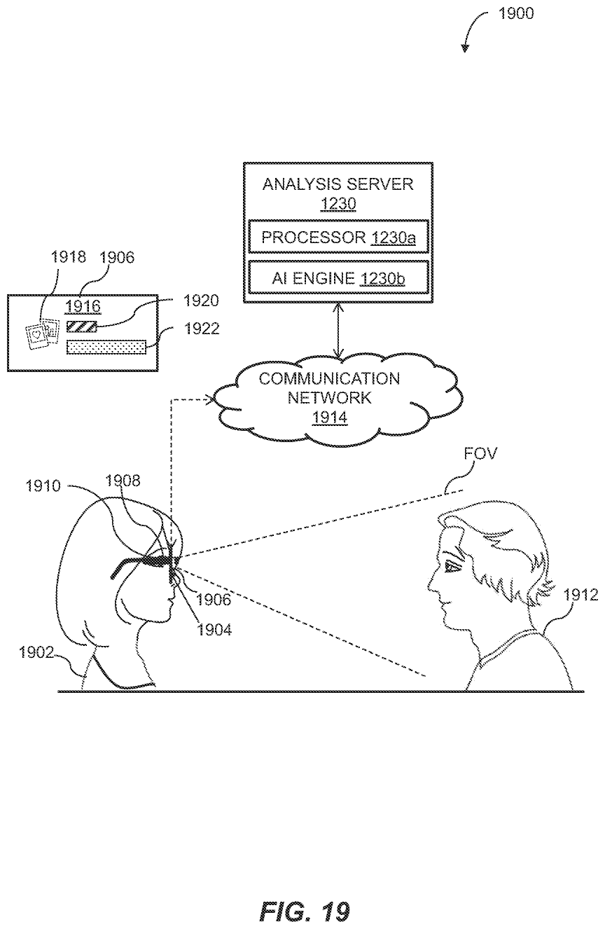

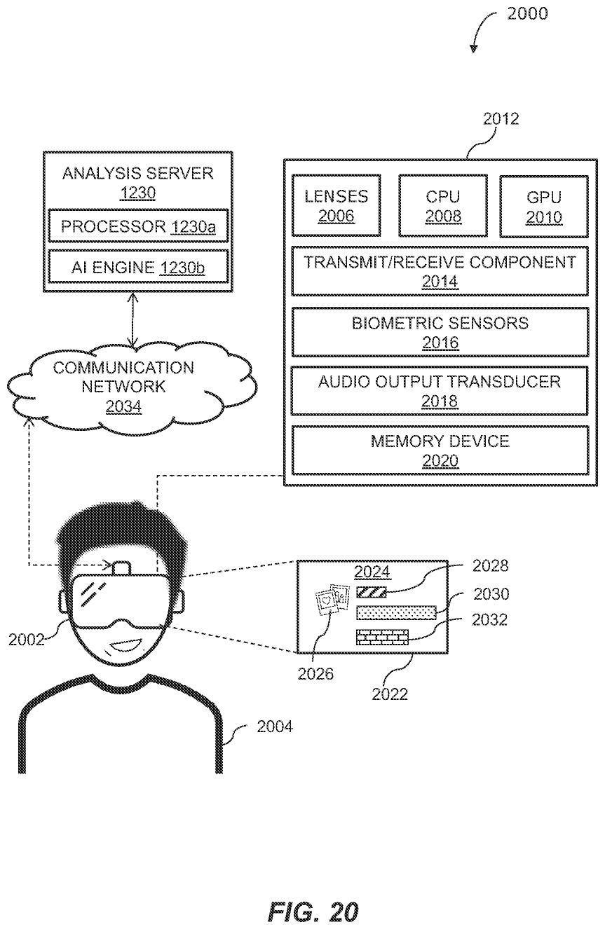

In view of the foregoing, and by way of an additional example, FIG. 18 shows aspects of a method 1800 or methods for controlling progress of audio-video content, such as social interaction or immersive content experience, based on sensor data of multiple users, composite neuro-physiological state and/or content engagement power. FIG. 18 is described in conjunction with the foregoing figures and FIGS. 19 and 20, as described hereinafter. FIG. 19 shows a system 1900 corresponding to a social interaction between two users and FIG. 20 shows a system 2000 corresponding to viewing an immersive content, such as a game, a narrative, or a virtual social interaction, by a user.

In an aspect, the social interaction of multiple users may be supported by a social interaction application that may be one of a dating application or a social media app. The multiple users may be addressed as participants that may include a first user who may be interacting with one or more second users, either personally or virtually. In an embodiment, the social interaction application may be launched at an electronic device associated with the first user. In such embodiment, the social interaction application may correspond to a personal app for the first user to find the best match amongst the one or more second users. In another embodiment, the social interaction application may be launched at a smart device, such as an IPTV, associated with a viewer or an audience. In such embodiment, the social interaction application may correspond to one of a live event streaming app, an on-demand app, or a broadcasting app meant for the viewers or the audience for entertainment purposes.

Accordingly, the method 1800 may be performed by a variety of electronic devices, such as a mixed reality output device, a non-immersive flat screen device, a projector, an electronic device (such as a wearable device or a mobile device) or other output devices including a programmable computer, one or more computers in communication with the output device, or a combination of an output device and one or more computers in communication with the output device.

In another aspect, an immersive content experience emphasizes immersion and interactivity with immersive content. The immersive content may be categorized as one of a spatial immersive content, a strategic immersive content, a narrative immersive content, or a tactical immersive content that supports multiple users. In accordance with various non-limiting examples, the immersive content may be one of a card game, a bluffing game, an action video game, an adventure video game, a role-playing video game, interactive polls and quizzes, animated data visualizations or infographics, 3D images and video, a simulation video game, a strategy video game, a sports video game and a party video game.

Accordingly, the method 1800 may be performed by a variety of AR and VR devices, or other head-mounted display or headset immersive visual output devices that include a programmable computer, in communication with one or more external devices, such as gaming controllers or other accessories.

Referring to FIG. 18, a computer-implemented method for controlling progress of social interaction and immersive content experience based on sensor data of multiple users, composite neuro-physiological state and/or content engagement power may include, at 1810, monitoring at least one of a personal interaction of the first user or audio-video content displayed on the output unit of the electronic device of the first user. Audio-video content may be associated with digital data representing a social interaction of the first user or a user engagement of the first user with the immersive content displayed at the output unit.

By way of a first example illustrated in FIG. 19, the first user may be a lead dater 1902 using a wearable device, such as smart glasses 1904. The smart glasses 1904 may comprise various electronic components, such as an output unit (for example, the screen unit 1906), biometric sensors 1908, and communication components 1910.

In an aspect, the lead dater 1902 may be involved in a personal interaction with a second user, such as a first candidate dater 1912. The first candidate dater 1912 may be in a field-of-view (FOV) of the one or more sensors, i.e., the biometric sensors 1908, positioned at the smart glasses 1904 worn by the lead dater 1902. Neuro-physiological responses of the first user, i.e., the lead dater 1902, and the second user, i.e., the first candidate dater 1912, may be sensed using the biometric sensors 1908 of the wearable device, i.e., the smart glasses 1904. The client device 300, i.e., the smart glasses 1904, may convert sensor signals from the biometric sensors 1908 into biometric data and send the biometric data to the analysis server 1230 via the communication components 1910 and the communication network 1914.

It should be noted that the smart glasses 1904 is an example of the client device 300 (FIG. 3) in a specific form factor. The client device 300 may be provided in various other form factors as well, of which the smart glasses 1904 provides but one example.

By way of a second example illustrated in FIG. 20, the first user may be a player actor 2004 using a wearable device, such as an immersive VR stereoscopic display device 2002, in an immersive environment, such as virtual live theatre or robotic interactive theatre, facilitated by an artificial intelligence (AI) engine 1230b of the analysis server 1230.

The immersive VR stereoscopic display device 2002 may be designed to be worn close to the face of the player actor 2004, enabling a wide field of view using a small screen size, such as in a smartphone, and providing stereoscopic display output for a realistic perception of 3D space for the player actor 2004. When wearing the immersive VR stereoscopic display device 2002, the player actor 2004 may view a display though a pair of lenses 2006 driven by a CPU 2008 and/or a GPU 2010, via an internal bus of a display and communications unit 2012. Components of the display and communications unit 2012 may further include, for example, a transmit/receive component 2014. Such transmit/receive component 2014 may enable wireless communication between the CPU 2008 and an external server, such as the analysis server 1203, via a wireless coupling facilitated by the communication network 2034. The transmit/receive component 2014 may operate using any suitable high-bandwidth wireless technology or protocol, including, for example, cellular telephone technologies such as 3rd 4th or 5th Generation Partnership Project (3GPP) Long Term Evolution (LTE) also known as 3G, 4G, or 5G, Global System for Mobile communications (GSM) or Universal Mobile Telecommunications System (UMTS), and/or a wireless local area network (WLAN) technology for example using a protocol such as Institute of Electrical and Electronics Engineers (IEEE) 802.11. The transmit/receive component 2014 or components may enable streaming of video data to the display and communications unit 2012 from a local or remote video server, and uplink transmission of sensor and other data to the local or remote video server for control or audience response techniques as described herein.

Components of the display and communications unit 2012 may further include one or more sensors, for example, biometric sensors 2016 coupled to the CPU 2008 via a communications bus. Such biometric sensors 2016 may include, for example, an accelerometer/inclinometer array providing orientation data for indicating an orientation of the display and communications unit. As the display and communications unit 2012 is fixed to the head of the player actor 2004, this data may also be calibrated to indicate an orientation of the head of the player actor 2004. The biometric sensors 2016 may further include, for example, a GPS sensor indicating a geographic position of the player actor 2004. The biometric sensors 2016 may further include, for example, a camera or image sensor positioned to detect an orientation of the eyes of the player actor 2004, or to capture video images of the physical environment (for VR mixed reality) of the player actor 2004, or both. In some embodiments, a camera, image sensor, or other sensor configured to detect eyes or eye movements may be mounted in a support structure and coupled to the CPU via the bus and a serial bus port (not shown), for example, a USB or other suitable communications port. The biometric sensors 2016 may further include, for example, an interferometer positioned in the support structure and configured to indicate a surface contour to the eyes of the player actor 2004. The biometric sensors 2016 may further include, for example, a microphone, an array of microphones, or other audio input transducer for detecting spoken user commands or verbal and non-verbal audible reactions to display output. The biometric sensors 2016 may include subvocalization mask using electrodes may determine subvocalized words that may be used as command input, as indications of arousal or valence, or both.

Components of the display and communications unit 2012 may further include, for example, an audio output transducer 2018, for example a speaker or a piezoelectric transducer, or an audio output port for headphones or other audio output transducer mounted in headgear or the like. The audio output transducer 2018 may provide surround sound, multichannel audio, so-called ‘object-oriented audio’, or other audio track output accompanying stereoscopic immersive VR video display content. Components of the display and communications unit 2012 may further include, for example, a memory device 2020 coupled to the CPU 2008 via a memory bus. The memory device 2020 may store, for example, program instructions that when executed by the processor, i.e., the CPU 2008, cause the apparatus to perform operations as described herein. The memory device 2020 may also store data, for example, audio-video data in a library or buffered during streaming from a network node.

In an aspect, the player actor 2004 may be involved in a virtual or immersive interaction with one or more second users, such as non-programmable characters (NPCs), that are computer-controlled characters. In other words, the NPCs may be controlled by the AI engine 1230b of the analysis server 1230. The NPCs may be in the FOV of the player actor 2004 in the immersive environment. The virtual interaction may correspond to the player actor 2004 actively interacting with the NPCs and/or participating in a game or a social experience via an avatar or other agency. Neuro-physiological responses of the first user, i.e., the player actor 2004, may be sensed using biometric sensors 2016 of the wearable device, i.e., the immersive VR stereoscopic display device 2002. Further, emotions of the second user, i.e., a first NPC, may be inferred using corresponding software solutions provided by the CPU 2008 for detecting emotive conjugates, such as Russel conjugates, from the speech signals. Additionally, various actions, inactions, and body language of the second user, i.e., a first NPC, may be detected using various computer visions and analytic techniques executed at the GPU 2010.

The client device 300, i.e., the immersive VR stereoscopic display device 2002, may convert signals from the biometric sensors 2016, the CPU 2008, and the GPU 2010 into player actor data and NPC data. The client device 300, i.e., the immersive VR stereoscopic display device 2002, may further send the player actor data and the NPC data to the analysis server 1230 via the transmit/receive component 2014 and the communication network 2034.

The immersive VR stereoscopic display device 2002 may be another example of the client device 300 (FIG. 3) in a specific form factor. The client device 300 may be provided in various other form factors as well, of which the immersive VR stereoscopic display device 2002 provides but one example.

Having described examples of suitable clients, servers, and networks for performing signal processing of biometric sensor data for detection of neuro-physiological state in communication enhancement applications, more detailed aspects of suitable signal processing methods will be addressed.

The method 1800 may include, at 1820, receiving sensor data from one or more sensors positioned on the electronic device of the first user to sense neuro-physiological responses of the first user and the one or more second users. The one or more sensors positioned on the electronic device of the first user may operate in a bidirectional or multidirectional mode to sense the sensor data, i.e., the neuro-physiological data, of the first user and the one or more second users. Non-limiting examples of the sensor data may include one or more of electroencephalographic (EEG) data, galvanic skin response (GSR) data, facial electromyography (fEMG) data, electrocardiogram (EKG) data, video facial action unit (FAU) data, brain machine interface (BMI) data, video pulse detection (VPD) data, pupil dilation data, functional magnetic imaging (fMRI) data, output of Russel conjugate analysis data, functional near-infrared data (fNIR), phased array radar (PAR) data, phased array microphone (PAM) data, and time-division multiple access (TDMA) data.

By way of the first example illustrated in FIG. 19, the wearable device, i.e., the smart glasses 1904, worn by the first user, i.e., the lead dater 1902, may further include the biometric sensors 1908. In accordance with various implementations, the biometric sensors 1908 may be embedded within, or integrated with other components positioned at the smart glasses 1904. Non-limiting examples of the biometric sensors 1908 may include one or more of an EEG, a GSR, an fEMG sensor, an EKG, a FAU, a BMI, a VPD, a pupil dilation sensor, a fMRI data, a Russel conjugate detector, an fNIR sensor, a PAR, a PAM, and a TDMA sensor. The sensor data, for example neuro-physiological responses, corresponding to the lead dater 1902 and the first candidate dater 1912 may be received and analysed at the analysis server 1230. The analysis server 1230 may be communicatively connected with the communication components 1910 of the smart glasses 1904, via the communication network 1914. In such manner, the analysis server 1230 may receive biometric data from the biometric sensors 1908 positioned on the client device 300, i.e., the smart glasses 1904, of the lead dater 1902 to sense neuro-physiological responses of the first user, i.e., the lead dater 1902, and the second user, i.e., the first candidate dater 1912, via the communication network 1914.

By way of the second example illustrated in FIG. 20, the wearable device, i.e., the immersive VR stereoscopic display device 2002 worn by the first user, i.e., the player actor 2004, may include the biometric sensors 2016, the CPU 2008, and the GPU 2010. In accordance with various implementations, the biometric sensors 2016, the CPU 2008, and the GPU 2010 may be embedded within, or integrated with other components positioned at the immersive VR stereoscopic display device 2002. Non-limiting examples of the biometric sensors 1908 may include one or more of electrodes or microphone to sense heart rate, a temperature sensor configured for sensing skin or body temperature, an image sensor coupled to an analysis module to detect facial expression or pupil dilation, a microphone to detect verbal and nonverbal utterances, or other biometric sensors for collecting biofeedback data including nervous system responses capable of indicating emotion via algorithmic processing, including any sensor as already described in connection with FIG. 3 at the sensor 328. Other non-limiting examples of the biometric sensors may include one or more of an EEG, a GSR, an fEMG sensor, an EKG, a FAU, a BMI, a VPD, a pupil dilation sensor, a fMRI data, a Russel conjugate detector, an fNIR sensor, a PAR, a PAM, and TDMA sensor.

The player actor data (for example neuro-physiological responses, corresponding to the player actor 2004) and the NPC data (corresponding to the first NPC) may be received and analysed at the analysis server 1230. The analysis server 1230 may be communicatively connected with the transmit/receive component 2014 of the immersive VR stereoscopic display device 2002, via the communication network 2034.

The method 1800 may include, at 1830, determining, based on the sensor data of the first user and the one or more second users, at least one of a CNS value for the social interaction application and a CEP value for the immersive content. The sensor data from the one or more sensors may include any one or more of the data described herein for arousal, valence, or other measures. In an embodiment, the analysis server 1230 may be configured to determine the CNS value and the CEP value using the arousal, valence, or other measures.

In an aspect, the CEP value may be determined at least in part by determining arousal values based on the sensor data and comparing a stimulation average arousal based on the sensor data with an expectation average arousal. Further, the expectation average arousal may be determined based on further sensor data measuring a like involuntary response of the first user while engaged with known audio-video stimuli. The known audio-video stimuli comprising a known non-arousing stimulus and a known arousing stimulus may be played. The CEP value may be determined at least in part by detecting one or more stimulus events based on the sensor data exceeding a threshold value for a time period. One of multiple event powers may be calculated for each user and for each stimulus event and the event powers may be aggregated.

Accordingly, weights may be assigned to each event power based on one or more source identities for the sensor data. The expectation average arousal may be determined at least in part by detecting one or more stimulus events based on the further sensor data exceeding a threshold value for a time period and calculate one of multiple expectation powers for the known audio-video stimuli for each user and for each stimulus event. Accordingly, the CEP power may be calculated at least in part by calculating a ratio of the sum of the event powers to an aggregate of the expectation powers. In other words, the CEP value may be calculated based on a ratio of a sum of the event powers to the expectation power for a comparable event in corresponding genre.

In another aspect, a digital representation of valence values may be determined based on the sensor data. The digital representation of the valence values may be normalized based on like values collected for a known audio-video stimuli. A valence error measurement may be determined based on comparing the digital representation of the valence values to a targeted valence value for at least one of a social interaction and a targeted emotional arc for the immersive content experience. The targeted valence value may be set based on input from the user.

In a similar manner, with reference to the list additional operations 1400 and 1500 in FIGS. 14 and 15 respectively, the CNS value may be calculated based on a ratio of a sum of the event powers to the expectation power for a comparable event in a social interaction. It should be noted that the elements of the operations for calculating the CEP and CNS values may be performed in any operative order, and any one or any number of them may be omitted.

By way of the first example illustrated in FIG. 19, the analysis server 1230 may receive the sensor data of the first user, i.e., the lead dater 1902, and the one or more second users, i.e., the first candidate dater 1912, and determine the CNS value for the social interaction application when the social interaction application corresponds to a personal app. However, in case the social interaction is being viewed by the viewer or the audience as one of a live streamed content, an on-demand content or a broadcast content, the analysis server 1230 may further determine the CEP value of the audio-video content of the social interaction.

By way of the second example illustrated in FIG. 20, the analysis server 1230 may receive the player actor data of the first user, i.e., the player actor 2004, and the NPC data of the one or more second users, i.e., the NPCs, and determine the CNS value based on a social experience of the player actor 2004 with the NPCs via an avatar or other agency. The analysis server 1230 may further calculate the CEP value of the immersive content, such as a VR game.

In an aspect, the CEP value and the CNS value or other neuro-physiological measure correlated to the social interaction or the immersive content experience may be recorded in a computer memory. In another aspect, the CEP value and the CNS value or other neuro-physiological measure may be indicated to the first user and/or a recipient. In yet another aspect, the progress of the at least one of the social interaction and the immersive content experience may be controlled based on the CEP value and the CNS value or other neuro-physiological measure, thus determined.

The method 1800 may include, at 1840, predicting, by the processor 1230a in conjunction with the AI engine 1230b, one or more recommendations for one or more action items for the first user. The one or more recommendations may be predicted, using machine learning tools, based on the sensor data of the first user and the one or more second users, and the at least one of the CNS value and the CEP value.

By way of the first example illustrated in FIG. 19, the analysis server 1230 may receive the sensor data from the client device 300, such as the smart glasses 1904, and determine the neuro-physiological state of the lead dater 1902 and the first candidate dater 1912 based on the valence/arousal neuro-physiological model where valence is positive/negative and arousal is magnitude, as illustrated in FIG. 7A. From such model, producers of the social interaction application and other creative productions may verify the intention of the social experience by measuring social theory constructs such as tension (hope vs. fear) and rising tension (increase in arousal over time) and more. During the social interaction mediated through the social interaction application, an algorithm may use the neuro-physiological model for operation of the social interaction application dynamically based on the psychological state or predisposition of the lead dater 1902 and the first candidate dater 1912. For example, based on various types of the sensor data of the lead dater 1902 and the first candidate dater 1912, such as heart rates, emotional states, skin responses showing arousal, calories burned, pupil dilation/focus, electrodermal activity, and the like, in addition to the CNS value, the analysis server 1230 may predict a first connection genuineness score based on the social interaction between lead dater 1902 and the first candidate dater 1912. On similar lines, the analysis server 1230 may further predict additional connection genuineness scores based on the social interactions between lead dater 1902 and the other candidates. Accordingly, based on the highest connection genuineness score, such as the first connection genuineness score, the analysis server 1230 may recommend the best blind date, i.e., the first candidate dater 1912, for the lead dater 1902.

By way of another example, the analysis server 1230 may receive the sensor data from another instance of the client device 300, such as the AR glasses, and determine the neuro-physiological state of a performer, such as a stand-up comedian, and the audience based on the valence/arousal neuro-physiological model where valence is positive/negative and arousal is magnitude, as illustrated in FIG. 7A. In a situation, when the content, such as jokes, of the performer, i.e., the comedian, is falling flat, i.e., the CEP value of the content indicates that the targeted emotional state in cohort is not met, the analysis server 1230 may predict, using machine learning tools, narrative or speech elements likely to produce the targeted emotional state in the cohort. The analysis server 1230 may recommend the predicted narrative or speech elements to the performer, which may be projected on the display unit of the AR glasses.

By way of the second example illustrated in FIG. 20, the analysis server 1230 may receive the sensor data from the client device 300, such as the immersive VR stereoscopic display device 2002. In one aspect, the analysis server 1230 may determine the CEP value for the immersive content based on the sensor data of the player actor 2004. In another aspect, the analysis server 1230 may determine the neuro-physiological state, or the CNS value, of the interaction between the player actor 2004 and the NPCs based on the valence/arousal neuro-physiological model. Accordingly, the analysis server 1230 may predict one or more recommendations for one or more action items for the player actor 2004. A recommendation may correspond to a selection of a branch having a combination of elements scored as most likely to produce the targeted emotional response. In addition, the analysis server 1230 may base the branching decision partly on a direct input of the player actor 2004 in a manner resembling an interactive video game, by weighing the direct input together with emotional indicators. Examples of the direct input may include, for example, spoken or texted verbal input, input from a game controller, bodily movement detected by a camera array, or selection of control links in a user interface. Further, the analysis server 1230 may base the branching decision partly on contextual indicators, such as dialog with the NPCs or other player actors.

The method 1800 may include, at 1850, creating a feedback loop based on the sensor data of the first user and the one or more second users, the at least one of the CNS value and the CEP value, and the predicted one or more recommendations. The analysis server 1230 may transmit the feedback loop, thus created, to the client device 300 for rendering. At the client device 300, the content of the feedback loop may be rendered at an output unit, such as the display device 320, of the electronic device of the first user during play of the at least one of the social interaction application and the immersive content experience. In accordance with an embodiment, the predicted one or more recommendations may be rendered on the output unit of the electronic device of the first user during play of the at least one of the social interaction application and the immersive content experience.

Referring to FIG. 3, the display device 320 may be coupled to the processor 302, for example via the graphics processing unit 318 integrated in the processor 302 or in a separate chip. In various aspects, the display device 320 may be incorporated into the smart glasses 1904 (FIG. 19), the immersive VR stereoscopic display device 2002 (FIG. 20), the AR glasses, or may be a mobile device, computer monitor, home theater or television screen, or projector in a screening room or theater. In a real social interaction application, the first user may avoid using a display in favor of audible input through an earpiece or the like, or tactile impressions through a tactile suit. In virtual social interaction applications, video output driven by a mixed reality display engine operating on the processor 302, or other application for coordinating user inputs with an immersive content display and/or generating the display, may be provided to the display device 320 and output as a video display to the first user. Similarly, the amplifier/speaker or other audio output transducer 316 may be coupled to the processor 302 via the audio processor 312. Audio output correlated to the video output and generated by the media player module 308, a video game or other application facilitating social interaction or other application may be provided to the audio transducer 316 and output as audible sound to the first user. The audio processor 312 may receive an analog audio signal from the microphone 314 and convert it to a digital signal for processing by the processor 302. The microphone may be used as a sensor for detection of neuro-physiological (e.g., emotional) state and as a device for user input of verbal commands, or for social verbal responses to the one or more second users.

Further, once the predicted one or more recommendations are rendered on the output unit of the electronic device, the progress of the at least one of the social interaction and the immersive content experience may be controlled by the first user. The first user may perform one or more action items based on the predicted one or more recommendations such that the progress of the at least one of the social interaction and the immersive content experience indicates greater realism, interest, fulfilment, and enjoyment of the social experience or game play.

By way of example of the dating application, as described further in FIG. 19, once the lead dater 1902 has interacted with other blind dates as well, the analysis server 1230 may determine that the first connection genuineness score corresponding to the first candidate dater 1912 is the highest or exceeds a threshold value, whereas other connection genuineness scores corresponding to other candidates is either lesser than the first connection genuineness score or lesser than the threshold value. Accordingly, the analysis server 1230 may predict that the first candidate dater 1912 is the best match for the lead dater 1902. Based on the predicted recommendation, the lead dater 1902 may look forward to having a relationship with the first candidate dater 1912. Other non-limiting examples of the controlled progress of the social interaction in case of the social interaction application may include at least one of selecting a new challenge for the first user, matching the first user to one of the one or more second users, intervention by the first user upon viewing a report and overriding the match, or determining capabilities of an avatar or another agency associated with each of the first user and the one or more second users.

In an aspect, the client device 300, i.e., the smart glasses 1904, may output the measures via output devices, such as the screen unit 1906, for example a display screen, as a graphical display 1916 or other useful format (e.g., audible output). The graphical display 1916 may display a report on neuro-physiological state measures for conversation sequence statements or groups of statements. For example, the graphical display 1916 may present data on emotional affect alongside the photo or video 1918 of the first candidate dater 1912 while the lead dater 1902 and the first candidate dater 1912 continue with their conversation. The data may further include an indication of an arousal value 1920 and a valence value 1922. In an aspect, the system 1900 may use a CNS model for communication where CNS values may be presented and measured alongside a conversation.

The system 1900 may provide an alert any time an increase in the arousal value 1920 and an associated increase in the valence value 1922 is observed. The alert may then be appraised by the human for interpretation.

In a related example, the social interaction application may provide the audience various options of presence in the viewing experience. In an aspect, the audience may enjoy a traditional lean back viewing without any impact which may highlight selects from the biometric data from the one or more sensors. In another aspect, the audience may choose to interact with data streams to watch the physiological responses unfold and see if the connections are being made in real-time.

In yet another aspect, viewers from the audience may have an option to select their personal favourite amongst the various candidate blind dates. The personal favourite may indicate a candidate dater the viewers want the lead dater 1902 to go out with again. In case the viewers' choice is someone neither the lead dater 1902 nor the first candidate dater 1912 that the analysis server 1230 has selected, the viewers may receive an input (audio or visual) that may explain the reasoning behind why the other candidate daters were not chosen. The viewers may then see the process play out to find out if the lead dater 1902 selects on his/her own or allows the analysis server 1230 to select and see the other candidate dates and its result.

In yet another aspect, the viewers may be able to play trivia to learn about dating and relationships from the analysis server 1230. Corresponding voting choices and trivia results may then be shared on social media.

In yet another aspect, the analysis server 1230 may be launched as a live AI engine on social media platforms to share dating and relationship facts. Accordingly, the analysis server 1230 may continuously engage with the viewers, answer their questions, share the interactions, and build community and experiences around the social interaction application across multiple platforms including streaming, social media, VR, and the metaverse.

In yet another aspect, the social interaction application may allow the viewers an external agency in casting and selection process. The viewers may download the social interaction application, and register themselves for consideration to be a dater on a viewing content (that is being streamed or broadcasted), and we can incorporate such audition footage in future episodes. The viewers may also vote on such crowdsourced casting options for who they think should be daters, and be able to vote on which people should get second dates. Thus, audience engagement in the viewing content may be increased by offering participation and stakes as they watch the show to see how the dates turn out.

In yet another aspect, the analysis server 1230 may observe a group of people that are dating each other throughout a season to determine the most compatible couple. The analysis server 1230 may facilitate the couples who may begin dating other people, while being able to watch their significant other's dates and data in real-time. Further, the analysis server 1230 may enable the viewers to follow one lead dater through a season-long dating journey through a pool of prospective daters.

By way of example of the VR game, as described further in FIG. 20, the analysis server 1230 may determine that the player actor 2004 is actively interacting with an NPC (which is in the FOV of the player actor 2004) during the play of the VR game, such as a card game, a bluffing game, an action video game, an adventure video game, a role-playing video game, a simulation video game, a strategy video game, a sports video game or a party video game. Accordingly, the analysis server 1230 may predict a recommendation for the player actor 2004.

In an aspect, the analysis server 1230 may store target story arcs based on the VR game or other application facilitating social interaction of the player actor 2004 with the NPCs. Such target story arcs may be stored in the memory device 2020 as a sequence of targeted values in any useful neuro-physiological model for representing user neuro-physiological state in a social interaction, for example the valence/arousal neuro-physiological model. Using the example of a valence/arousal neuro-physiological model, the analysis server 1230 may perform a difference calculation to determine the error between the planned/predicted and measured arousal and valence. The analysis server 1230 may predict a recommendation based on determined error.

In an aspect, once a delta between the predicted and measured values passes a threshold, then the analysis server 1230 may recommend a branching action. For example, if the valence of the player actor 2004 is in the ‘wrong’ direction based on the game design, then the analysis server 1230 may change the content, for example select a new challenge for the player actor 2004, by the following logic:

If absolute value of (Valence Predict—Valence Measured)>0 then change the content. The change in content may be several different items specific to what the software has learned about the player actor 2004 or it may be a trial or recommendation from an AI process.
Likewise, if the arousal error falls below a threshold (e.g. 50%) of predicted (Absolute value of (error)>0.50*Predict) then the analysis server 1230 may change the content.

Based on such recommendation, the player actor 2004 may perform an action item, for example, accept the new challenge. As a result, the analysis server 1230 may determine that the absolute value of (Valence Predict—Valence Measured) is now close to zero and the arousal error now exceeds the threshold. Such updated error values may indicate that the player actor 2004 is finding the VR game or the social experience more realistic, interesting, fulfilling, and amusing. Other non-limiting examples of the controlled progress of the immersive content experience may include at least one of determining a winner, changing a parameter setting for audio-visual game output, selecting a new challenge for the first user, or determining capabilities of an avatar associated with each of the first user and the one or more second users, or a non-player character.

In another aspect, the analysis server 1230 may predict a recommendation pertaining to a change the characteristics or behaviours of characters, objects, or environments appearing in cinematic content of the VR game or the social interaction, with or without altering the narrative. The analysis server 1230 may select characteristics and behaviours of audio-video elements based on emotional indicators, predictions of emotional response, and a targeted emotional arc for the player actor 2004. Accordingly, the analysis server 1230 may predict responses to changes and weigh emotional inputs with user inputs, using techniques that parallel branch selection. For example, past responses of the player actor 2004 may indicate an association between a theme (such as unicorn) and positive arousal and valence values. Accordingly, for scenes intended to be happy, the analysis server 1230 may cause more objects to be displayed in accordance with the preferred theme in the virtual environment for the player actor 2004.

In yet another aspect, the analysis server 1230 may guide a group of daters from around the world. The AI engine 1230b of the analysis server 1230 may facilitate avatars of such group of daters who may interact in a VR environment and forge real connections without knowing the true appearances. The winning couple may ultimately meet one another in the real world after building a relationship that spans space and time by occurring in virtual reality.

In accordance with an embodiment, the client device 300, i.e., the immersive VR stereoscopic display device 2002, may output the measures via output devices, such as a screen unit 2022, for example a display screen, as a graphical display 2024 or other useful format (e.g., audible output). The graphical display 2024 may display the photo/video 2026 of one or more NPCs and a report on neuro-physiological state measures for conversation sequence statements or groups of statements. For example, the graphical display 2024 may include an indication of arousal value 2028, valence value 2030 and content engagement power value 2032. The system 2000 may provide an alert any time a rapid increase in the arousal value 2028 and an associated increase in the valence value 2030 is observed. The system 2000 may further provide an alert any time there's a rapid decrease in the content engagement power value 2032. The alert may then be appraised by the human for interpretation.

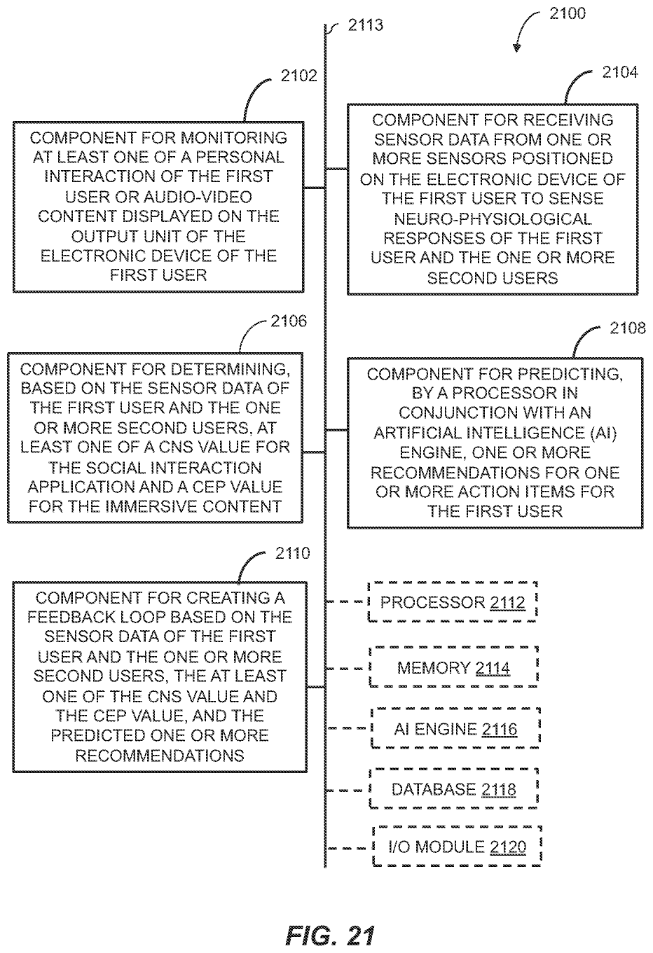

FIG. 21 is a conceptual block diagram illustrating components of an apparatus or system 2100 for controlling progress of audio-video content based on sensor data of multiple users, composite neuro-physiological state and/or content engagement power. The apparatus or system 2100 may include additional or more detailed components for performing functions or process operations as described herein. For example, the processor 2112 and memory 2114 may contain an instantiation of a process for calculating CNS and CEP in real time as described herein above. As depicted, the apparatus or system 2100 may include functional blocks that can represent functions implemented by a processor, software, or combination thereof (e.g., firmware).

As illustrated in FIG. 21, the apparatus or system 2100 may comprise an electrical component 2102 for monitoring, by at least one processor, at least one of a personal interaction of the first user or audio-video content displayed on the output unit of the electronic device of the first user. The component 2102 may be, or may include, a means for said monitoring. Said means may include the processor 2112 coupled to the memory 2114, the processor 2112 executing an algorithm based on program instructions stored in the memory 2114. Such algorithm may include, for example, detecting a context of the social interaction or the immersive content experience, including that the social interaction or the immersive content experience is directed to eliciting a targeted neuro-physiological response, and creating an association between the social interaction or the immersive content experience and the targeted response.

The system 2100 may further include an electrical component 2104 for receiving sensor data from one or more sensors positioned on the electronic device of the first user to sense neuro-physiological responses of the first user and the one or more second users. The component 2104 may be, or may include, a means for said receiving. Said means may include the processor 2112 coupled to the memory 2114, the processor 2112 executing an algorithm based on program instructions stored in the memory 2114. Such algorithm may include a sequence of more detailed operations, for example, configuring a data port of the I/O module 2120 to receive sensor data from the one or more sensors, configuring a connection to the one or more sensors, receiving digital data at the port, and interpreting the digital data as the sensor data.

The system 2100 may further include an electrical component 2106 for determining, based on the sensor data of the first user and the one or more second users, at least one of a CNS value for the social interaction application and a CEP value for the immersive content. The component 2106 may be, or may include, a means for said determining. Said means may include the processor 2112 coupled to the memory 2114, the processor 2112 executing an algorithm based on program instructions stored in the memory 2114.

The system 2100 may further include an electrical component 2108 for predicting one or more recommendations for one or more action items for the first user. The component 2108 may be, or may include, a means for said predicting. Said means may include the processor 2112 and the AI engine 2116 coupled to the memory 2114 and the database 2118, the processor 2112 and the AI engine 2116 executing an algorithm based on program instructions and data stored in the memory 2114 and the database 2118, respectively. Such algorithm may include a sequence of more detailed operations, for example, encoding the CNS and CEP values and storing the encoded values in a computer memory, i.e., the memory 2114, or sending the encoded value to an output device, such as the output unit of the electronic device of the first user, for presentation to the first user.

The system 2100 may further include an electrical component 2110 for creating a feedback loop based on the sensor data of the first user and the one or more second users, the at least one of the CNS value and the CEP value, and the predicted one or more recommendations. The component 2108 may be, or may include, a means for said creating. Said means may include the processor 2112 and the AI engine 2116 coupled to the memory 2114 and the database 2118, the processor 2112 and the AI engine 2116 executing an algorithm based on program instructions and data stored in the memory 2114 and the database 2118, respectively. Such algorithm may include a sequence of more detailed operations, for example, creation of feedback loops and controlling a progress of the at least one of the social interaction and the immersive content experience is controlled by the first user based on the predicted one or more recommendations rendered on the output unit of the electronic device of the first user. The content of the feedback loop may be rendered on the output unit of the electronic device of the first user during play of the at least one of the social interaction application and the immersive content experience.

The system 2100 may optionally include a processor module having at least one processor 2112. The processor 2112 may be in operative communication with the modules 2102-2110 via a bus 2113 or similar communication coupling. In the alternative, one or more of the modules may be instantiated as functional modules in a memory of the processor 2112. The processor 2112 may initiate and schedule the processes or functions performed by electrical components 2102-2110.

In related aspects, the system 2100 may include the I/O module 2120 or an equivalent network interface module operable for communicating with system components over a computer network. The network interface module may be, or may include, for example, an Ethernet port or serial port (e.g., a Universal Serial Bus (USB) port), a Wi-Fi interface, or a cellular telephone interface.

In further related aspects, the system 2100 may optionally include a module for storing information, such as, for example, a memory 2114. The computer readable medium or the memory 2114 may be operatively coupled to the other components of the system 2100 via the bus 2113 or the like. The memory 2114 may be adapted to store computer readable instructions and data for effecting the processes and behaviour of the modules 2102-2110, and subcomponents thereof, or the processor 2112, the method 1800 and one or more of the additional operations disclosed herein, or any method for performance by a media player described herein. The memory 2114 may retain instructions for executing functions associated with the modules 2102-2110. While shown as being external to the memory 2114, it is to be understood that the modules 2102-2110 can exist within the memory 2114 or an on-chip memory of the processor 2112.

In various embodiments, the processor 2112 may include networked microprocessors from devices operating over a computer network. In addition, the system 2100 may connect to an output device as described herein, via the I/O module 2120 or other output port.

Those of skill would further appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the application and design constraints imposed on the overall system. Skilled artisans may implement the described functionality in varying ways for each application, but such implementation decisions should not be interpreted as causing a departure from the scope of the present disclosure.

As used in this application, the terms “component”, “module”, “system”, and the like are intended to refer to a computer-related entity, either hardware, a combination of hardware and software, software, or software in execution. For example, a component or a module may be, but are not limited to being, a process running on a processor, a processor, an object, an executable, a thread of execution, a program, and/or a computer. By way of illustration, both an application running on a server and the server can be a component or a module. One or more components or modules may reside within a process and/or thread of execution and a component or module may be localized on one computer and/or distributed between two or more computers.

Various aspects will be presented in terms of systems that may include several components, modules, and the like. It is to be understood and appreciated that the various systems may include additional components, modules, etc. and/or may not include all the components, modules, etc. discussed in connection with the figures. A combination of these approaches may also be used. The various aspects disclosed herein can be performed on electrical devices including devices that utilize touch screen display technologies, heads-up user interfaces, wearable interfaces, and/or mouse-and-keyboard type interfaces. Examples of such devices include VR output devices (e.g., VR headsets), AR output devices (e.g., AR headsets), computers (desktop and mobile), televisions, digital projectors, smart phones, personal digital assistants (PDAs), and other electronic devices both wired and wireless.

In addition, the various illustrative logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device (PLD) or complex PLD (CPLD), discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Operational aspects disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, digital versatile disk (DVD), Blu-ray™, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a client device or server. In the alternative, the processor and the storage medium may reside as discrete components in a client device or server.

Furthermore, the one or more versions may be implemented as a method, apparatus, or article of manufacture using standard programming and/or engineering techniques to produce software, firmware, hardware, or any combination thereof to control a computer to implement the disclosed aspects. Non-transitory computer readable media can include but are not limited to magnetic storage devices (e.g., hard disk, floppy disk, magnetic strips, or other format), optical disks (e.g., compact disk (CD), DVD, Blu-ray™ or other format), smart cards, and flash memory devices (e.g., card, stick, or other format). Of course, those skilled in the art will recognize many modifications may be made to this configuration without departing from the scope of the disclosed aspects.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

In view of the exemplary systems described supra, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

What is claimed is:

1. A system, comprising:
a memory for storing instructions; and
a processor configured to execute the instructions, and based on the executed instructions,
the processor is further configured to:
receive sensor data from one or more sensors positioned on an electronic device of a first user to sense neuro-physiological responses of the first user and one or more second users,
wherein the one or more second users are in field-of-view (FOV) of the one or more sensors positioned on the electronic device of the first user, and
wherein the first user and the one or more second users are participants of at least one of a social interaction application or an immersive content experience;
determine, based on the sensor data of the first user and the one or more second users, at least one of a composite neurological state (CNS) value for the social interaction application and a content engagement power (CEP) value for immersive content;
predict, by the processor in conjunction with an artificial intelligence (AI) engine, one or more recommendations for one or more action items for the first user based on the sensor data of the first user and the one or more second users, and the at least one of the CNS value and the CEP value; and
create a feedback loop based on the sensor data of the first user and the one or more second users, the at least one of the CNS value and the CEP value, and the predicted one or more recommendations,
wherein content of the feedback loop is rendered on an output unit of the electronic device of the first user during play of the at least one of the social interaction application and the immersive content experience, and
wherein a progress of the at least one of a social interaction and the immersive content experience is controlled by the first user based on the predicted one or more recommendations rendered on the output unit of the electronic device of the first user.

2. The system according to claim 1, wherein the sensor data comprises one or more of electroencephalographic (EEG) data, galvanic skin response (GSR) data, facial electromyography (fEMG) data, electrocardiogram (EKG) data, video facial action unit (FAU) data, brain machine interface (BMI) data, video pulse detection (VPD) data, pupil dilation data, functional magnetic imaging (fMRI) data, output of Russel conjugate analysis, functional near-infrared data (fNIR), phased array radar (PAR) data, phased array microphone (PAM) data, and time-division multiple access (TDMA) data.

3. The system according to claim 1, wherein the processor is further configured to monitor at least one of a personal interaction the first user or audio-video content displayed on the output unit of the electronic device of the first user,
wherein the audio-video content is associated with digital data representing a social interaction of the first user or a user engagement of the first user with immersive content displayed at the output unit.

4. The system according to claim 1, wherein the at least one of the CNS value and the CEP value is determined using arousal values,
wherein the arousal values are based on the sensor data and comparison of a stimulation average arousal with an expectation average arousal, and
wherein the stimulation average arousal is based on the sensor data.

5. The system according to claim 4, wherein the processor is further configured to:
detect one or more stimulus events based on the sensor data exceeding a threshold value for a time period;
determine the expectation average arousal based on sensor data measuring a like involuntary response of a user while engaged with a known audio-video stimuli;
calculate one of multiple event powers for each of the first user and the one or more second users and for each of the stimulus events and aggregating the event powers;
assign weights to each of the event powers based on one or more source identities for the sensor data; and
calculate an expectation power for the known audio-video stimuli for the first user and the one or more second users and for each of the stimulus events.

6. The system according to claim 5, wherein the processor is further configured to calculate the CNS value based on a ratio of a sum of the event powers to the expectation power for a comparable event in a social interaction.

7. The system according to claim 5, wherein the processor is further configured to calculate the CEP value based on a ratio of a sum of the event powers to the expectation power for a comparable event in corresponding genre.

8. The system according to claim 1, wherein the processor is further configured to:
determine a digital representation of valence values based on the sensor data; and
normalize the digital representation of the valence values based on like values collected for a known audio-video stimuli.

9. The system according to claim 8, wherein the processor is further configured to determine a valence error measurement based on comparing the digital representation of the valence values to a targeted valence value for at least one of a social interaction and a targeted emotional arc for the immersive content.

10. The system according to claim 1, wherein the social interaction application is one or more of a dating application or a social networking application, and
wherein the progress of the social interaction application corresponds to at least one of: selecting a new challenge for the first user, matching the first user to one of the one or more second users, intervention by the first user upon viewing a report and overriding the match, or determining capabilities of an avatar associated with each of the first user and the one or more second users.

11. The system according to claim 1, wherein the immersive content is categorized as a spatial immersive content, strategic immersive content, narrative immersive content, or tactical immersive content,
wherein the immersive content is one of a card game, a bluffing game, an action video game, an adventure video game, a role-playing video game, a simulation video game, a strategy video game, a sports video game and a party video game, and
wherein the progress of the immersive content corresponds to at least one of: determining a winner, changing a parameter setting for audio-visual game output, selecting a new challenge for the first user, or determining capabilities of an avatar associated with each of the first user and the one or more second users, or a non-player character.

12. A method, comprising:
receiving, by a processor, sensor data from one or more sensors positioned on an electronic device of a first user to sense neuro-physiological responses of the first user and one or more second users,
wherein the one or more second users are in field-of-view (FOV) of the one or more sensors positioned on the electronic device of the first user, and
wherein the first user and the one or more second users are participants of at least one of a social interaction application or an immersive content experience;
determining, by the processor, at least one of a composite neurological state (CNS) value for the social interaction application and a content engagement power (CEP) value for immersive content based on the sensor data of the first user and the one or more second users;
predicting, by the processor in conjunction with an artificial intelligence (AI) engine, one or more recommendations for one or more action items for the first user based on the sensor data of the first user and the one or more second users, and the at least one of the CNS value and the CEP value; and
creating, by the processor, a feedback loop based on the sensor data of the first user and the one or more second users, the at least one of the CNS value and the CEP value, and the predicted one or more recommendations,
wherein content of the feedback loop is rendered on an output unit of the electronic device of the first user during play of the at least one of the social interaction application and the immersive content experience,
wherein a progress of the at least one of a social interaction and the immersive content experience is controlled by the first user based on the predicted one or more recommendations rendered on the output unit of the electronic device of the first user.

13. The method according to claim 12, further comprising monitoring at least one of a personal interaction the first user or audio-video content displayed on the output unit of the electronic device of the first user,
wherein the audio-video content is associated with digital data representing a social interaction of the first user or a user engagement of the first user with immersive content displayed at the output unit.

14. The method according to claim 12, wherein the at least one of the CNS value and the CEP value is determined using arousal values,
wherein the arousal values are based on the sensor data and comparison of a stimulation average arousal with an expectation average arousal, and
wherein the stimulation average arousal is based on the sensor data.

15. The method according to claim 14, further comprising:
detecting, by the processor, one or more stimulus events based on the sensor data exceeding a threshold value for a time period;
determining, by the processor, the expectation average arousal based on sensor data measuring a like involuntary response of a user while engaged with a known audio-video stimuli;
calculating, by the processor, one of multiple event powers for each of the first user and the one or more second users and for each of the stimulus events and aggregating the event powers;
assigning, by the processor, weights to each of the event powers based on one or more source identities for the sensor data; and
calculating, by the processor, an expectation power for the known audio-video stimuli for the first user and the one or more second users and for each of the stimulus events.

16. The method according to claim 15, further comprising calculating the CNS value based on a ratio of a sum of the event powers to the expectation power for a comparable event in a social interaction.

17. The method according to claim 15, further comprising calculating, by the processor, the CEP value based on a ratio of a sum of the event powers to the expectation power for a comparable event in corresponding genre.

18. The method according to claim 12, further comprising:
determining, by the processor, a digital representation of valence values based on the sensor data; and
normalizing, by the processor, the digital representation of the valence values based on like values collected for a known audio-video stimuli.

19. The method according to claim 18, further comprising determining, by the processor, a valence error measurement based on comparing the digital representation of the valence values to a targeted valence value for at least one of a social interaction and a targeted emotional arc for the immersive content experience.

20. A non-transitory computer readable medium, having stored thereon, computer executable code, which when executed by a processor, cause the processor to execute operations, the operations comprising:
receiving sensor data from one or more sensors positioned on an electronic device of a first user to sense neuro-physiological responses of the first user and one or more second users,
wherein the one or more second users are in field-of-view (FOV) of the one or more sensors positioned on the electronic device of the first user, and
wherein the first user and the one or more second users are participants of at least one of a social interaction application or an immersive content experience;
determining at least one of a composite neurological state (CNS) value for the social interaction application and a content engagement power (CEP) value for immersive content based on the sensor data of the first user and the one or more second users;
predicting, by an artificial intelligence (AI) engine, one or more recommendations for one or more action items for the first user based on the sensor data of the first user and the one or more second users, and the at least one of the CNS value and the CEP value; and
creating a feedback loop based on the sensor data of the first user and the one or more second users, the at least one of the CNS value and the CEP value, and the predicted one or more recommendations,
wherein content of the feedback loop is rendered on an output unit of the electronic device of the first user during play of the at least one of the social interaction application and the immersive content experience,
wherein a progress of the at least one of a social interaction and the immersive content experience is controlled by the first user based on the predicted one or more recommendations rendered on the output unit of the electronic device of the first user.
